(19) United States
(12) Patent Application Publication
(10) Pub. No.: US 2006/0134073 A1
(43) Pub. Date: Jun. 22, 2006

(54) Compositions and methods for ex vivo preservation of blood vessels for vascular grafts using analogues of cAMP and cGMP

(76) Inventors: Yoshifumi Naka, Cliffside Park, NJ (US); David J. Pinsky, Ann Arbor, MI (US); David M. Stern, Augusta, GA (US)

Correspondence Address: WILMER CUTLER PICKERING HALE AND DORR LLP;COLUMBIA UNIVERSITY, 399 PARK AVENUE, NEW YORK, NY, 10020, US

(21) Appl. No.: 11/173,537

(22) Filed: Jul. 1, 2005

Related U.S. Application Data
(60) Provisional application No. 60/631,107, filed on Nov. 24, 2004.

(51) Int. Cl. C12N 5/08; A61K 35/14; A61K 31/7076; A61K 31/52

(52) U.S. Cl. 424/93.7; 435/366; 514/46; 514/263.24

(57) ABSTRACT

The present invention relates to ex vivo methods for preserving/maintaining blood vessels that are to be used as vascular grafts. The present invention also relates to compositions comprising an analogue of cAMP and/or an analogue of cGMP for use in the methods of the invention, which compositions are free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α.

The present application claims benefit under 35 U.S.C. §119(e) of U.S. Provisional Application No. 60/631,107 filed Nov. 24, 2004, the disclosure of which is incorporated by reference herein in its entirety.

The invention disclosed herein was made with U.S. Government support from the National Heart, Lung, and Blood Institute/National Institutes of Health (Grant Nos. HL04484-01 and RO1-HL63967). Accordingly, the U.S. Government has certain rights in this invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

### 1. FIELD OF THE INVENTION

The present invention provides methods for the use of adenosine 3′,5′-cyclic monophosphate (cAMP) and/or guanosine 3′,5′-cyclic monophosphate (cGMP) analogues in the preservation/maintenance of blood vessels, such as arteries or veins, that are to be used as vascular grafts prior to attachment to the recipient blood vessel in vascular surgical procedures. The present invention also provides compositions comprising an analogue of cAMP and/or an analogue of cGMP for use in ex vivo preservation/maintenance of blood vessels useful as vascular grafts, which compositions are free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of tumor necrosis factor-α (TNF-α).

### 2. BACKGROUND OF THE INVENTION

2.1 Organ Preservation Solutions

Adequate preservation of organs intended for transplantation is critical to the proper functioning of the organ following implantation. Long preservation times are desired to enable cross-matching of donor and recipient to improve subsequent survival, as well as to allow for coast to coast and international transportation of organs to expand the donor and recipient pools. Organ preservation requires preservation of both the structure and function of the organ, including the specialized cells of the organ as well as the blood vessels and other cells found in the organ that together are responsible for its function.

Many different organ preservation solutions have been designed, as investigators have sought to lengthen the time that an organ may remain extra-corporeally, as well as to maximize function of the organ following implantation. Several of the key solutions that have been used over the years include: 1) the Stanford University solution [see, e.g., Swanson et al., 1988, Journal of Heart Transplantation, 7(6):456-467); 2) a modified Collins solution [see, e.g., Maurer et al., 1990, Transplantation Proceedings, 22(2):548-550; Swanson et al., supra]; and 3) the University of Wisconsin solution (U.S. Pat. No. 4,798,824, issued to Belzer et al.). Further, U.S. Pat. Nos. 5,552,267 and 5,370,989 to Stern et al. and a publication by Kayano et al., 1999, J. Thoracic Cardiovascular Surg. 118:135-144 describe an organ preservation solution known as the Columbia University solution.

In addition to the composition of the organ preservation or maintenance solution, the method of organ preservation also affects the success of preservation. Several methods of cardiac preservation have been studied in numerous publications: 1) warm arrest/cold ischemia; 2) cold arrest/macroperfusion; 3) cold arrest/microperfusion; and 4) cold arrest/cold ischemia. The first method involves arresting the heart with a warm cardioplegic solution prior to explantation and cold preservation, but this method fails because of the rapid depletion of myocardial energy store during the warm period. The second method, which involves arresting the heart with a cold preservation solution, is better; but continuous perfusion of the heart with preservation solution during the storage period fails because of the generation of toxic oxygen radicals. In addition, the procedure of the second method is cumbersome and does not lend itself to easy clinical use. The third method, called “trickle perfusion”, is better but also cumbersome. The fourth method of preservation is that of a cold cardioplegic arrest followed by a period of cold immersion of the heart. The fourth method is currently the standard method of cardiac preservation. This fourth method reliably preserves hearts for periods of up to six hours, but less than four hours is considered ideal for this method.

2.2 Coronary Artery Bypass Graft (CABG)

Coronary artery disease is a major medical problem throughout the world. Coronary arteries, as well as other blood vessels, frequently become clogged with plaque, impairing the efficiency of the heart's pumping action, and inhibiting blood flow which can lead to heart attack and death. In certain instances, these arteries can be unblocked through relatively noninvasive techniques such as balloon angioplasty. In other cases, a bypass of the blocked vessel is necessary.

A coronary artery bypass graft (“CABG”) involves performing an anastomosis on a diseased coronary artery to reestablish blood flow to an ischemic portion of the heart muscle. Improved long-term survival has been demonstrated bypassing the left anterior descending artery with a left internal mammary artery and this encouraged surgeons to extend revascularization with arterial grafts to all coronary arteries.

Since the internal mammary artery can only be used for two CABG procedures (using right and left internal mammary arteries, respectively), where multiple-vessels need to be bypassed, other arteries or veins have to be used. Such other arteries or veins that have been used include the right gastroepiploic artery, the inferior epigastric artery, the internal mammary artery (also known as the internal thoracic artery), the radial artery, and the saphenous vein. The internal mammary artery is the most common arterial conduit used for CABG; yet, despite its widespread use and superior patency when compared to the saphenous vein (Grondin et al., 1984, Circulation, 70 (suppl I): 1-208-212; Camereon et al., 1996, N Engl J Med, 334: 216-219), the saphenous vein continues to be one of the most popular conduits for CABG (Roubos et al., 1995, Circulation, 92 (9 Suppl) 1131-6).

During a typical coronary artery bypass graft procedure using the saphenous vein, a section of the saphenous vein is surgically removed from the leg and the graft is retained ex vivo (out of the body) for a length of time prior to attachment to another blood vessel within the body (Angelini and Jeremy, 2002, Biorheology, 39 (3-4): 491-499). In a bypass operation involving such a venous graft, the graft is harvested by a surgically invasive procedure from the leg of the patient and then stored for up to about four hours ex vivo as the heart surgery is conducted. Although there are variations in methodology in surgical preparation of the heart, the first part of the procedure typically requires an incision through the patient's sternum (sternotomy), and in one technique, the patient is then placed on a bypass pump so that the heart can be operated on while not beating. In alternative techniques, the heart is not stopped during the procedure. Having harvested and stored the saphenous vein or arterial blood vessel conduit and upon completion of the surgery to prepare the heart for grafting, the bypass procedure is performed. A precise surgical procedure is required to attach the bypass graft to the coronary artery (anastomosis), with the graft being inserted between the aorta and the coronary artery. The inserted venous/arterial segments/transplants act as a bypass of the blocked portion of the coronary artery and thus provide for a free or unobstructed flow of blood to the heart. More than 500,000 bypass procedures are performed in the United States every year.

The overall short and long term success of the CABG procedure is dependent on several factors including the condition of the graft that is to be inserted which itself depends on any form of damage during the removal of the graft from the body or deterioration or damage of the graft due to storage conditions. In such circumstances, the short term detrimental effect can be potentially lethal thrombotic disease as a result of contact of flowing blood with a changed phenotype of the graft due to its deterioration or damage during the removal or storage stage. Possible long term detrimental effects include the vein graft itself becoming diseased, stenosed, or occluded, similar to the original bypassed vessel. In this case, the diseased or occluded saphenous vein grafts are associated with acute ischemic syndromes necessitating some form of intervention. It is, therefore, of critical importance not only that care be taken in the surgical procedure to remove the blood vessel to be used as the graft in surgical bypass procedures including CABG, but, also that no deterioration or damage occurs in the storage period of the graft prior to attachment to another blood vessel and the resumption of blood flow in that vessel.

In order to decrease the likeliness of short and long term detrimental consequences of grafting blood vessels in surgical procedures, including coronary arterial bypass grafting (CABG), and consequently to improve the overall outcome of patients undergoing such procedures, there is a need for improved storing conditions for such vascular grafts during the time period from harvesting of the graft to attaching the graft to another blood vessel in the patient.

Citation or identification of any reference in Section 2 or in any other section of this application shall not be construed as an admission that such reference is available as prior art to the present invention.

### 3. SUMMARY OF THE INVENTION

The present invention is directed to a solution comprising an analogue of adenosine 3′,5′-cyclic monophosphate (cAMP) and/or an analogue of guanosine 3′,5′-cyclic monophosphate (cGMP) for preserving a blood vessel, or a functional portion thereof, removed from an individual, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of tumor necrosis factor-α (TNF-α). The blood vessel or portion thereof can be used as a vascular graft in the same or different individual. It is believed that the use of an analogue of cAMP and/or cGMP enhances the viability of the blood vessel and will result in less damage or phenotypic change of the blood vessel as a result of storage conditions. Thus, it is believed that blood vessels so treated will improve short and long term outcomes of vascular bypass procedures involving blood vessel grafts, including, but not limited to, coronary artery bypass, abdominal aneurysm repair, carotid endarterectomy, deep vein occlusion, or popliteal aneurysm repair.

Accordingly, the present invention provides a solution comprising an analogue of adenosine 3′,5′-cyclic monophosphate (cAMP) and/or an analogue of guanosine 3′,5′-cyclic monophosphate (cGMP) and heparinized blood, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of tumor necrosis factor-α (TNF-α). Alternatively, the present invention provides a solution consisting of an analogue of cAMP and/or an analogue of cGMP and heparinized blood, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α. The present invention also provides a method of preserving a blood vessel comprising contacting an isolated blood vessel or portion thereof ex vivo with a solution comprising an analogue of cAMP and/or an analogue of cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α. The present invention also provides a method of preserving a blood vessel or a functional portion thereof comprising contacting an isolated blood vessel or portion thereof ex vivo with a solution consisting of (i) a fluid; and (ii) an analogue of cAMP and/or an analogue of cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α, said fluid selected from the group consisting of heparinized blood, buffered saline with or without heparin, Ringer's saline with or without heparin, the modified Columbia University solution, the Euro-Collins solution, the University of Wisconsin solution, the low-potassium dextran glucose solution and the CELSIOR™ solution.

In other embodiments of the present invention, an isolated ex vivo blood vessel or functional portion thereof in contact with a solution comprising an analogue of cAMP and/or an analogue of cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α, is provided. As used herein, a functional portion of a blood vessel is a portion of a blood vessel of sufficient size as to be able to act as a vascular graft.

The present invention also provides a container containing a blood vessel or functional portion thereof in contact with a solution comprising an analogue of cAMP and/or an analogue of cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α.

Further, the present invention provides a method of using a blood vessel as a vascular graft comprising contacting an isolated blood vessel or functional portion thereof ex vivo with a solution comprising an analogue of cAMP and/or an analogue of cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α; and inserting the blood vessel into a patient so as to form a vascular graft in the patient. The present invention also provides a method for performing a coronary artery bypass graft in a patient comprising removing from contact with a blood vessel or functional portion thereof a solution comprising an analogue of cAMP and/or an analogue of cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α; and grafting the blood vessel or functional portion thereof into the patient so as to serve as a coronary bypass graft.

Analogues of cAMP and cGMP are well known in the art. Exemplary analogues of cAMP or cGMP that can be used according to the invention are those that have modifications to the purine ring system, to the ribose, or to the phosphate group. See FIGS. 1a-1b showing the chemical structure of cAMP and the subparts of the molecule, i.e., phosphate group, purine ring, imidazole ring, pyrimidine ring. Preferred analogues of cAMP useful in the present invention include, but are not limited to, dibutyryl adenosine 3′,5′-cyclic monophosphate (db cAMP), 8-bromo-adenosine 3′,5′-cyclic monophosphate (8-bromo cAMP) Rp-adenosine 3′,5′-cyclic monophosphate (Rp-cAMP) and Sp-adenosine 3′,5′-cyclic monophosphate (Sp-cAMPS) (the S isomer of cAMP). Preferred analogues of cGMP useful in the present invention include, but are not limited to, dibutyryl guanosine 3′,5′-cyclic monophosphate (db cGMP), 8-bromo-guanosine 3′,5′-cyclic monophosphate (8-bromo cGMP), 8-(4-chlorophenylthio)-guanosine 3′,5′-cyclic monophosphate (8-(4-chlorophenylthio) cGMP, Rp-guanosine 3′,5′-cyclic monophosphate (Rp-cGMP) and Sp-guanosine 3′,5′-cyclic monophosphate (Sp-cGMPS) (the S isomer of cGMP).

In a specific embodiment, a preservation solution of the invention also does not contain an inhibitor of Type V phosphodiesterase. In a specific embodiment, the solution does not contain (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, (v) an inhibitor of Type V phosphodiesterase, and (vi) an inhibitor of TNF-α, and optionally does not contain the foregoing in an amount greater than any endogenous amount of (i) the inhibitor of Type I phosphodiesterase, (ii) the inhibitor of Type II phosphodiesterase, (iii) the inhibitor of Type III phosphodiesterase, (iv) the inhibitor of Type IV phosphodiesterase, (v) the inhibitor of Type V phosphodiesterase, and (vi) the inhibitor of TNF-α, respectively, normally found in human blood.

In an alternative embodiment of the present invention, the solution comprises nitroglycerin, optionally with an analogue of cAMP and/or cGMP of the present invention. In one embodiment, the nitroglycerin is present in a concentration range of about 0.01 mg/ml to about 10 mg/ml. In a particular embodiment, the concentration is about 0.1 mg/ml. In another embodiment, the solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α. In yet another embodiment, the solution comprising nitroglycerin lacks an analogue of cAMP and/or cGMP.

The present invention is also directed to kits comprising (a) in a first container, a blood vessel preservation solution; and (b) a first plurality of vials, each vial comprising a lyophilized aliquot of an analogue of cAMP and/or an analogue of cGMP.

### 4. BRIEF DESCRIPTION OF THE FIGURES

FIGS. 1a-1b. FIG. 1a shows the chemical structure of adenosine 3′,5′-cyclic monophosphate (cAMP) and specifically points out the purine ring system and the ribose moiety of the molecule. FIG. 1b shows the chemical structure of the purine ring system of cAMP and the imidazole and pyrimidine ring sub-parts of the purine ring system.

FIGS. 2a-2b. Photograph (2a) and schematic representation (2b) demonstrating the procedures of the vein patch implantation model. A 5 mm long vein segment taken from the right external jugular vein was sutured onto the abdominal aorta after preservation (IVC, Inferior Vena Cava; Ao, Aorta).

FIGS. 3a-3k. FIGS. 3a-3k are photomicrographs of cross sections of jugular vein-abdominal aorta composite vessels. Sections are oriented with vein patch on top and artery segment on bottom. Elastic Van-Gieson stain for postoperative 21-day samples shows elastic lamina of the arterial wall, muscle and collagen (3a). White arrows indicate sutures approximately corresponding to the junction of vein patch (black arrows) and arterial wall. Masson trichrome stain for postoperative 21-day samples shows the extracellular matrix and the cellular portion (3b). Immunohistologic staining for PECAM-1 (CD31) (3c-3e), MAC-1 (3f-3h), and ICAM-1 (3i-3k) for postoperative days 1 (3c, 3f, 3i), 7 (3d, 3g, 3j), and day 21 (33, 3h, 3k) is shown. (Original magnification 100×(3a and 3b) or 400×(3c-3k)).

FIGS. 4a-4f show the influence of perioperative ischemic injury on the development of neointimal hyperplasia of vein grafts. Elastic Van-Gieson-stained sections of jugular vein-abdominal aorta composite vessels 21 days after the operation are shown: FIG. 4a is a vein graft that had been implanted immediately after harvest, without an interventing preservation period; FIG. 4b is a vein graft subjected to 2 hours of preservation in heparinized saline prior to implantation (Original magnification is 100×); FIG. 4c is a schematic representation of cross-section of the composite vessel. Statistical data of the total neointimal cell number (4d), neointimal area (4e) and percentage of neointimal expansion (4f) at postoperative day 21 are shown as means ±SEM for each group. Number of grafts were as follows: 15 in the nonpreservation group, 5 in the 1 hour preservation group, and 9 in the 2 hour preservation group. * P<0.05, ** P<0.01.

FIGS. 5a-5b. FIG. 5a shows cAMP contents in the harvested vein graft. FIG. 5b shows the effect of cAMP analogues (db-cAMP and 8-Br-cAMP) on neointimal expansion of vein grafts. The vein patch was stored in the heparinized saline without any additives (A); with cAMP analog db-cAMP (2 mmol/L, B); 8-Br-cAMP (0.1 mmol/L, C); or with 8-Br-cAMP (0.1 mmol/L) and the cAMP-dependent protein kinase inhibitor Rp-cAMPS (0.25 mmol/L, D). After 2 hours of preservation, the vein patch was implanted onto the abdominal aorta of same mouse and harvested at postoperative day 21. Data are shown as means ±SEM for each group. Number of grafts were as follows: A, 9; B, 9; C, 9; D, 8. * P<0.05, ** P<01.

FIGS. 6a-6g. FIGS. 6a-6g show leukocyte adhesion-infiltration to the vein patch. En face immunofluorescence demonstrated MAC-1⁺ cells adhering to the endothelium of the vein patch (a-c): a, vein graft without preservation; b, vein grafts subjected to 2 hours of preservation before implantation without preservation solution additive; c, grafts subjected to 2 hours of preservation with added cAMP analog db-cAMP. (Original magnification is 200×). d, MAC-1⁺ cells adhered to the surface of vein patch. Data are shown as means ±SEM for each group (n=10). Immunohistochemical staining demonstrates the presence of MAC-1⁺ cell infiltration in vein grafts at postoperative day 21(6e-6g). Original magnification is 400×.

FIG. 7 shows a Movat's Trichrome stain of a vein prior to grafting.

FIG. 8 shows a Movat's Trichrome stain of a vein which was placed for 1.0 hour in heparinized saline, grafted into an animal and explanted at 45 days.

FIG. 9 shows a Movat's Trichrome stain of a vein which was placed for 1.0 hour in PMA (positive control), grafted into an animal and explanted at 45 days.

FIG. 10 shows a Movat's Trichrome stain of a vein which was placed for 1.0 hour in 1.0 mM db-cAMP (experimental group), grafted into an animal and explanted at 45 days.

### 5. DETAILED DESCRIPTION OF THE INVENTION

It is believed that the use of an analogue of cAMP and/or an analogue of cGMP enhances the viability of a blood vessel and results in less damage or phenotypic change of the blood vessel as a result of storage conditions. Thus, blood vessels so treated should improve short and long term outcomes of vascular bypass procedures involving blood vessel grafts, including coronary artery bypass grafts, abdominal aneurysm repair, carotid endarterectomy, deep vein occlusion, and popliteal aneurysm repair. Exemplary blood vessels that can be so isolated include, but are not limited to, a saphenous vein, a mammary artery, a renal artery, and a radial artery.

5.1 Preservation Solutions

The present invention is directed to use of a solution comprising an analogue of cAMP and/or an analogue of cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of tumor necrosis factor-α (TNF-α). In specific embodiments, the solution can contain one analogue or more than one analogue of cAMP and/or cGMP. The solution can be an aqueous solution or a semi-solid gel. In a preferred embodiment, the solution is an aqueous solution. Any physiologic solution to which the analogue is added can be used in the present invention so long as the solution does not damage the blood vessel graft tissue that is placed in it. For example, the solution comprising the cAMP and/or cGMP analogue can be saline, buffered saline, phosphate buffered saline or Ringer's saline, each with or without heparin. The solution can also be Hank's Balanced Salt solution (HBSS), which typically comprises 1.26 mM CaCl₂, 5.36 mM KCl, 0.44 mM KH₂PO₄, 0.81 mM MgSO₄, 136 mM NaCl, 0.42 mM Na₂HPO₄, 6.1 mM glucose, 20 mM HEPES-NaOH, at pH 7.4 (Herreros et al., 2000, J. Neurochem 74(5):1941-1950) or modified HBSS, which typically comprises 143 mM NaCl, 5.6 mM KCl, 2 mM MgCl₂, 10 mM HEPES, 10 mM glucose, 0.2 mM CaCl₂, and 0.4% BSA, at pH 7.2 (Briddon et al., 1999, Blood 93:3847-3855). The solution can also be Ringer's Lactate, which typically comprises 155 mM NaCl, 5 mM KCl, 2 mM CaCl₂, 1 mM MgCl₂, 2 mM NaH₂PO₄, 10 mM HEPES, and 10 mM glucose, at pH 7.2 (Sturgill-Koszycki and Swanson, 2000, J Exp. Med. 192:1261-1272). The solution can also be Tyrodes buffer, which typically comprises 137 mM NaCl, 12 mM NaHCO₃, 26 mM KCl, 5.5 mM glucose, 0.1% BSA, and 5.0 mM Hepes at pH 7.35 (Kasirer-Friede et al., 2002, J. Biol. Chem., 277:11949-11956). The solution can also be Kreb's buffer, which typically comprises 119 mM NaCl, 4.7 mM KCl, 2.5 mM CaCl₂, 1.17 MgSO₄, 25 mM NaHCO₃, 1.18 mM KH₂PO₄, 0.026 mM EDTA, and 5.5 mM glucose (Knock et al., 2002, J. Physiology 538:879-890).

Optionally, the solutions of the present invention can also contain a variety of additional additives, such as sugars and preservatives, as detailed infra. In a preferred embodiment, the solution comprises the analogue of cAMP and/or cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α; and heparinized blood, or saline, buffered saline or Ringer's saline each with or without heparin, or the modified Columbia University solution, or the Euro-Collins solution, or the University of Wisconsin solution, or the low-potassium dextran glucose solution, or the CELSIOR™ solution. Alternatively, the solution consists of (i) a fluid; and (ii) an analogue of cAMP and/or cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α; said fluid selected from the group consisting of heparinized blood, or saline, buffered saline, or Ringer's saline each with or without heparin, the modified Columbia University solution, the Euro-Collins solution, the University of Wisconsin solution, the low-potassium dextran glucose solution, and the CELSIOR™ solution. In a preferred embodiment, the solution is sterilized.

In certain embodiments, the solutions of the present invention also comprise a sugar, for example, D-glucose, e.g., in an amount sufficient to support intracellular function and maintenance of cellular bioenergetics. In a preferred embodiment, the concentration of the sugar ranges from about 50 mM to about 80 mM. The solutions can also comprise magnesium ions, e.g., in an amount sufficient to support intracellular function and maintenance of cellular bioenergetics. In a preferred embodiment, the concentration of magnesium ions ranges from about 2 mM to about 10 mM. In particular, the magnesium ions can be derived from magnesium sulfate, magnesium gluconate, or magnesium phosphate, or suitable combinations thereof. The magnesium ions can also be derived from some other suitable magnesium containing compound. D-Glucose, adenosine, and magnesium ions are substrates for adenosine triphosphate (ATP) synthesis. Metabolic substrates such as D-glucose and perhaps adenosine for ATP formation are probably important for maintaining the small degree of anaerobic metabolism that occurs during ex vivo preservation of blood vessels. Basal energy metabolism (even during hypothermia) can be supported by the anaerobic metabolism of D-glucose. The presence of magnesium ion allows for the proper functioning of the enzymes needed for adenosine triphosphate (ATP) synthesis. In general, substrates for ATP synthesis are helpful to allow intracellular function and maintenance of cellular bioenergetics.

In other embodiments, the preservation solution also comprises a macromolecule of molecular weight greater than 20,000 daltons, e.g., in an amount sufficient to maintain endothelial integrity and cellular viability. In a preferred embodiment, the macromolecule of molecular weight greater than 20,000 daltons is a macromolecule having a molecular weight greater than about 100,000 daltons, a polysaccharide, or a polyethylene glycol. Other suitable macromolecules can be used. The macromolecule of molecular weight greater than 20,000 daltons can be a colloid. In a preferred embodiment, the polysaccharide is a dextran. Furthermore, in a preferred combination, the dextran is a dextran molecule having a molecular weight of 308,000 daltons. Macromolecules of molecular weight greater than 20,000 daltons are believed to be helpful in reducing trans-endothelial leakage and subsequent intracellular and interstitial edema in the reperfusion period, by serving to plug small endothelial leaks which may occur. Macromolecules may thus also prevent the extravasation of intravascular contents into the pericellular space, thus helping to prevent cellular swelling and rupture during the preservation and recovery periods.

The osmolarity of the preservation solution of the invention is also a factor in helping to prevent cellular swelling and rupture. The osmolarity of the solution should be greater than the cellular osmolarity. Cellular osmolarity is about 290 mOSm/l. In a preferred embodiment, the osmolarity ranges from about 315 mOSm/l to about 340 mOSm/l.

The preservation solution also optionally comprises potassium ions, preferably in a concentration greater than about 110 mM. The potassium ions can be derived from potassium sulfate, potassium gluconate, monopotassium phosphate (KH₂PO₄), or suitable combinations thereof. The potassium ions can also be derived from some other suitable potassium containing compound. In one embodiment, the concentration of potassium ions ranges from about 10 mM to about 165 mM. In another embodiment, the concentration of potassium ions ranges from about 110 mM to about 140 mM.

In other embodiments, the preservation solution also comprises a buffer in an amount sufficient to maintain the average pH of the solution during the period of blood vessel preservation at about the physiologic pH value. In a preferred embodiment, the buffer is monopotassium phosphate (KH₂PO₄). However, other suitable buffers may be used. The buffering capacity should be adequate to buffer the organic acids that accrue during ischemia. Because basal metabolism results in the generation of acid, preferably a buffering system is used. The pH of the solution can decline during prolonged storage times that can be employed with this solution. In a preferred embodiment, the initial pH of the preservation solution is adjusted to the alkaline side of normal physiologic pH because then the average pH during storage of the blood vessel in the preservation solution remains physiologic. Normal physiologic pH is about 7.4. A preferred embodiment of the preservation solution has a pH range of about 7.3 to about 7.6. The pH may be adjusted to the desired value with the addition of a suitable base, such as potassium hydroxide (KOH). Hence, during the period of preservation, the pH of the preservation solution starts on the alkaline side of physiologic pH, and may drift slowly down to the acidic side of physiologic pH. But the average pH of the preservation solution during the period of preservation is preferably about the physiologic value.

In other embodiments, the preservation solution may further comprise impermeant anions, e.g., in an amount sufficient to help maintain endothelial integrity and cellular viability. The impermeant anion can be the gluconate anion or the lactobionate anion. Other suitable impermeant anions can be used. In a preferred embodiment, the concentration of the gluconate anion ranges from about 85 mM to about 105 mM. The gluconate anion can be derived from potassium gluconate or magnesium gluconate. The gluconate anion can also be derived from some other suitable gluconate containing compound. Impermeant anions are large anions that cannot cross cell membranes, so that sodium is at least in part prevented from diffusing down its concentration gradient into the cell during the preservation period. Impermeant anions thus help to prevent cellular edema.

The preservation solution may further comprise an anticoagulant, e.g., in an amount sufficient to help prevent clotting of blood within the capillary bed of the blood vessel. The anticoagulant can be heparin or hirudin. Other suitable anticoagulants may be used. In a preferred embodiment, the concentration of heparin ranges from about 1000 units/l to about 100,000 units/l. Anticoagulants are believed to help in preventing clotting of blood within the capillary bed of the preserved blood vessel. Specifically, anticoagulants are believed to help prevent a total no-reflow phenomenon at the level of the microcirculation, which would be undesirable following re-implantation and could result in graft failure. Anticoagulants are believed to be helpful in ensuring that thrombosis does not occur during or after preservation, so that nutrient delivery and toxin removal can proceed.

In yet another embodiment, the preservation solution may further comprise an antioxidant, e.g., in an amount sufficient to help decrease reperfusion injury secondary to oxygen free radicals. The antioxidant can be butylated hydroxyanisole (BHA), butylated hydroxytoluene (BHT), Vitamin C, Vitamin E, or suitable combinations thereof. Other suitable antioxidants can be used. In a preferred embodiment, the antioxidant is butylated hydroxyanisole (BHA) at a concentration range from about 25 μM to about 100 μM, alone or in combination with butylated hydroxytoluene (BHT) at a concentration range from about 25 μM to about 100 μM. The preservation solution can further comprise a reducing agent, e.g., in an amount sufficient to help decrease reperfusion injury secondary to oxygen free radicals. Any suitable reducing agent can be used.

Optionally, the preservation solution may further comprise N-acetylcysteine in an amount sufficient to help cells produce glutathione. In a preferred embodiment, the concentration of N-acetylcysteine ranges from about 0.1 mM to about 5 mM. N-acetylcysteine is an agent which can enter cells and is believed to play a role in helping cells to produce glutathione, which is a reducing agent. During blood vessel preservation, glutathione is lost. Simply adding glutathione to the preservation solution, however, would likely be of little to no help, because it is now known that glutathione in solution does not enter easily into the cell.

In another optional embodiment, the preservation solution may further comprise an agent that helps prevent calcium entry into cells in an amount sufficient to help prevent calcium entry into cells. Agents that help prevent calcium entry into cells include so-called calcium channel blockers, as well as other agents that serve the described function. An agent that helps prevent calcium entry into cells that can be used is verapamil. Other suitable agents that help prevent calcium entry into cells may be used. In a preferred embodiment, the concentration of verapamil ranges from about 2 μM to about 25 μM. Agents that help prevent calcium entry into cells are believed to play a role in preventing calcium overload.

Optionally, the solution does not contain sodium. The absence of sodium in the solution is preferred, since any sodium which may enter the cells during the period of preservation (when energy currency is low and the normal trans-cellular gradient may not be well maintained) may 1) lead to cellular swelling, 2) cause calcium entry by facilitated diffusion (following re-implantation), and 3) sodium load the cell, such that a high amount of energy is required following reestablishment of blood flow before a normal membrane potential can be re-established.

In other embodiments, the preservation solution can further comprise a bacteriostat, in an amount sufficient to help inhibit the growth of, or destroy, bacteria. The bacteriostat can be cefazolin or penicillin. Other suitable bacteriostats or antibiotics can be used. In a specific embodiment, the concentration of cefazolin ranges from about 0.25 g/l to about 1 g/l. The addition of an antibiotic to the solution is a surgical consideration, due in one embodiment to the practical inability of sterilizing the solution completely, as the high molecular weight solutes would not pass through a 0.2 micron membrane filter which may be used in the preparation of the preservation solution. It is believed that gamma irradiation may be used to better sterilize the solution.

Optionally, the preservation solution further comprises a vasodilator in an amount sufficient to maintain vascular homeostasis. Preferably, the vasodilator is cell membrane permeable. The vasodilator can be selected from the group including, but not limited to, nitroglycerin, adenosine, and pertussis toxin. Suitable combinations of the vasodilators may be used. A solution of the invention contains a vasodilator, wherein the vasodilator is nitroglycerin and/or adenosine. In a specific embodiment, the concentration of nitroglycerin ranges from about 0.05 g/l to about 0.2 g/l. In another specific embodiment, the concentration of adenosine ranges from about 3 mM to about 20 mM.

In other embodiments, the solution can be any organ preservation solution known in the art in combination with an analogue of cAMP and/or an analogue of cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α. Illustrative examples of organ preservation solutions include, but are not limited to, the Euro-Collins solution, the University of Wisconsin solution, the low-potassium dextran glucose solution (PERFADEX™), the CELSIOR™ solution, and the modified Columbia University solution. In general, these solutions contain electrolytes and, optionally, sugars. One illustrative solution comprises a sugar in an amount sufficient to support intracellular function and maintenance of cellular bioenergetics, e.g., glucose, D-glucose; magnesium ions in an amount sufficient to support intracellular function and maintenance of cellular bioenergetics; potassium ions; and a buffer, e.g., a monopotassium phosphate or bicarbonate buffer, in an amount sufficient to maintain the average pH of the solution at about the physiologic pH value, i.e., about pH 7.3 to about pH 7.6. The specific composition of certain known solutions is listed below.

The Euro-Collins solution is described in Maurer et al., 1990, Transplantation Proceedings 22:548-550 and in Swanson et al., 1988, J. Heart Transplantation 7(6):456-467, and typically comprises 115 mM potassium, 10 mM sodium, 8 mM magnesium, 10 mM bicarbonate, 100 mM phosphate, and 120 mM glucose, at a pH of about 7.4 and with an osmolality of about 452 mOsm/L.

The University of Wisconsin solution is described in U.S. Pat. No. 4,798,824 to Belzer et al., and typically comprises 125 mM potassium, 30 mM sodium, 5 mM magnesium, 25 mM phosphate, 5 mM sulfate, 100 mM lactobionate, 50 mM hydroxyethyl starch, 5 mM adenosine and 1 mM allopurinol, at a pH of about 7.4 and with an osmolality of about 327 mOsm/L.

The low-potassium dextran glucose solution (PERFADEX™, commercially available from VitroLife, Gothenberg, Sweden), and typically comprises 4 mM potassium, 165 mM sodium, 2 mM magnesium, 101 mM chloride, 34 mM phosphate, 2 mM sulfate, 20 mM Dextran-40, and 56 mM glucose, at a pH of about 7.4 and with an osmolality of about 335 mOsm/L.

The CELSIOR™solution is commercially available from Genzyme, Cambridge, Mass., and typically comprises 60 mM mannitol, 80 mM lactobionic acid, 20 mM glutamic acid, 30 mM histidine, 0.25 mM calcium, 15 mM potassium, 13 mM magnesium, 100 mM sodium hydroxide, and 3 mM reduced glutathione, at a pH of about 7.3 and with an osmolality of about 320-360 mOsm/L.

The Columbia University solution is described in U.S. Pat. Nos. 5,370,989 and 5,552,267 to Stern et al., and typically comprises 120 mM potassium, 5 mM magnesium, 25 mM phosphate, 5 mM sulfate, 95 mM gluconate, 50 mM Dextran 50, 67 mM glucose, 5 mM adenosine, 2 mM dibutyryl adenosine 3′,5′-cyclic monophosphate (db cAMP), 0.1 mg/ml nitroglycerin, 50 μM butylated hydroxyanisole, 50 μM butylated hydroxytoluene, 0.5 mM N-acetylcysteine, 10 U/ml heparin, and 10 μM verapamil, at a pH of about 7.6 and with an osmolality of about 325 mOsm/L. As used herein, the modified Columbia University solution is the Columbia University solution lacking db cAMP. A preferred solution of the invention is the Columbia University solution. Another preferred solution of the invention is the modified Columbia University solution further comprising 8-bromo cAMP. Another preferred solution of the invention is the modified Columbia University solution further comprising Sp-cAMPS. Another preferred solution of the invention is the modified Columbia University solution further comprising 8-bromo guanosine 3′,5′-cyclic monophosphate (8-bromo cGMP). Another preferred solution of the invention is the modified Columbia University solution further comprising Sp-guanosine 3′,5′-cyclic monophosphate (Sp-cGMPS) (the S isomer of cGMP). Another preferred solution of the invention is the modified Columbia University solution further comprising 8-(4-chlorophenylthio)-guanosine 3′,5′-cyclic monophosphate (8-(4-chlorophenylthio) cGMP).

In a specific embodiment, a preservation solution of the present invention comprises, or alternatively consists of, an analogue of cAMP and/or an analogue of cGMP; a sugar in an amount sufficient to support intracellular function and maintenance of cellular bioenergetics; magnesium ions in an amount sufficient to support intracellular function and maintenance of cellular bioenergetics; a macromolecule of molecular weight greater than 20,000 daltons in an amount sufficient to maintain endothelial integrity and cellular viability; potassium ions in a concentration greater than about 110 mM; and a buffer in an amount sufficient to maintain the average pH of the blood vessel or portion thereof during said contacting step at about the physiologic pH value, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α. Optionally, the solution can further comprise a vasodilator such as adenosine, nitroglycerin and/or pertussis toxin. In one embodiment, the nitroglycerin is present in the preservation solution in a range of about 0.01 mg/ml to about 10 mg/ml.

In another specific embodiment, a preservation solution of the present invention comprises, or alternatively consists of, an analogue of cAMP and/or an analogue of cGMP; 67.4 mM D-glucose; 5 mM magnesium sulfate (MgSO₄); 25 mM monopotassium phosphate (KH₂PO₄); 50 g/l dextran (molecular weight 308,000 daltons); 95 mM potassium gluconate (K-gluconate); 50 μM butylated hydroxyanisole (BHA); 50 μM butylated hydroxytoluene (BHT); 0.5 mM N-acetylcysteine; 5 mM adenosine; 0.1 g/l nitroglycerin; 10 μM verapamil; 10,000 units heparin; and 0.5 g/l cefazolin, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α. The pH is adjusted to 7.6 with potassium hydroxide.

The amount of the preservation solution required in a surgical procedure, such as a cardiac arterial bypass graft (CABG) would be clear to one who is skilled in such surgical procedures, and depends, inter alia, upon the particular container used to hold the blood vessel and solution.

The preservation solution of the invention is suitable for use at room temperature, i.e., about 23-25° C. Alternatively, the preservation solution is suitable for use at the low temperatures that may be required or desired during vascular bypass, e.g., CABG, or other surgical procedure. For instance, temperatures of about 0.5 to about 10 degrees Centigrade, preferably 4° C., may be used for incubation in the preservation solution for CABG or other surgical procedure. In one embodiment, the preservation solution is used at a temperature in the range of about 10-25° C.

The present invention is also directed to a container containing a preservation solution of the present invention. In a preferred embodiment, the container is one of certain dimensions useful in preserving a blood vessel. In another embodiment, the container also contains the blood vessel or functional portion thereof, intended for use as a vascular graft, in contact with the solution.

In a specific embodiment, a preservation solution of the invention also does not contain an inhibitor of Type V phosphodiesterase. In a specific embodiment, the solution does not contain (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, (v) an inhibitor of Type V phosphodiesterase, and (vi) an inhibitor of TNF-α, and optionally does not contain the foregoing in an amount greater than any endogenous amount of (i) the inhibitor of Type I phosphodiesterase, (ii) the inhibitor of Type II phosphodiesterase, (iii) the inhibitor of Type III phosphodiesterase, (iv) the inhibitor of Type IV phosphodiesterase, (v) the inhibitor of Type V phosphodiesterase, and (vi) the inhibitor of TNF-α, respectively, normally found in human blood.

5.2 cAMP and cGMP Analogues

The preservation solutions of the present invention comprise an analogue of cAMP and/or analogue of cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α. In a specific embodiment, the analogue is present in the solution in a concentration range of about 0.05 mM to about 250 mM, preferably in a range of about 0.1 mM to about 100 mM. In an embodiment, the concentration range refers to the concentration of a single analogue in the solution. For example, in one embodiment wherein the solution comprises more than one analogue of cAMP and/or cGMP, the concentration of each analogue is about 250 mM in the solution. The optimal concentration of the analogue can be determined by standard techniques.

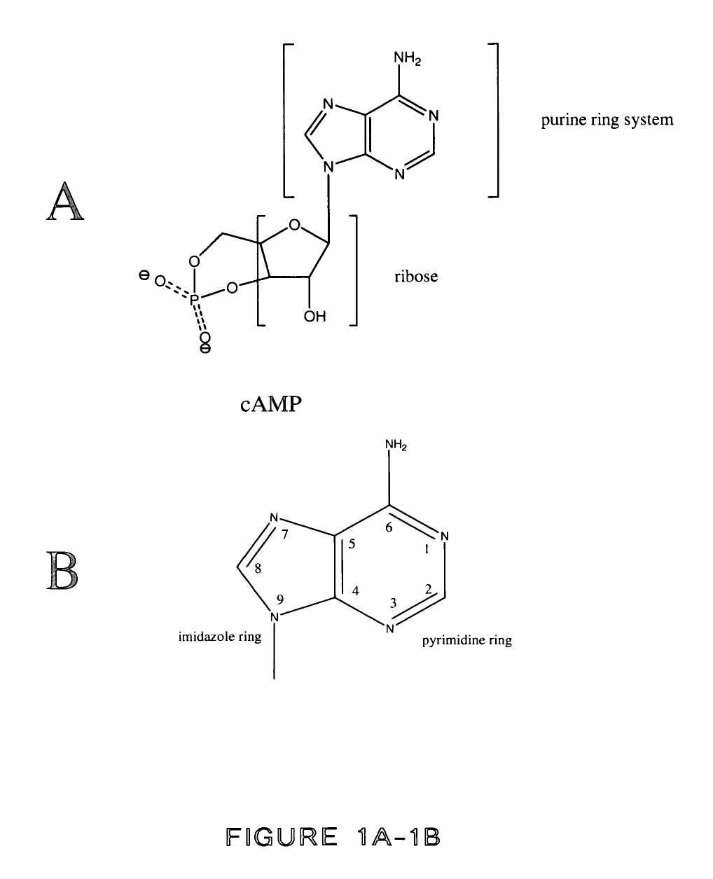

Analogues of cAMP and cGMP that can be used according to the invention are those that have modifications to the purine ring system, to the ribose, or to the phosphate group. See FIGS. 1A-1B showing the chemical structure of cAMP and the subparts of the molecule, i.e., phosphate group, purine ring, imidazole ring, pyrimidine ring. The purine ring system is the most commonly studied site for modification as it is essential for cyclic nucleotide recognition by its dependent kinase. Øgreid et al., 1985, Eur. J. Biochem. 150:219-227; Corbin et al., 1986, J. Biol. Chem. 261:1208-1214; Øgreid et al., 1989, Eur. J. Biochem. 181:19-31. Modifications to the purine ring system can be made in either the pyrimidine portion or the imidazole portion. For example, modifications to the pyrimidine portion of the ring system (positions 1, 2 or 6) alter binding affinity in direct correlation to the changes in tertiary structure or hydrophilic interactions; in contrast, modifications to the imidazole portion of the system (position 8) seem to regulate binding through a combination of electronic, steric and hydophobic forces. Corbin et al., 1986, J. Biol. Chem. 261:1208-1214. Although most substituents at position 8 reduce the affinity of the analog for its respective kinase, a few, notably 8-Br cAMP, have the opposite effect. Øgreid et al., 1989, Eur. J. Biochem. 181:19-31. This is thought due either to electronic effects in the case of electron withdrawing groups or the direct interaction of the substituent with the binding site. Corbin et al., 1986, J. Biol. Chem. 261:1208-1214.

Analogues of cAMP and cGMP also comprise simultaneous modifications to the purine ring system, the ribose or to the phosphate group. For example, modifications to the either the purine ring system or the ribose are often combined with a substitution of one of the exocyclic oxygens of the phosphate group by sulfur. Sulfur replacement at either the equatorial or axial position (Sp or Rp isomer, respectively) increases not only the lipophilicity of the compound but also induces its resistance to hydrolysis by phosphodiesterase. Braumann et al., 1985, J. Chromatogr. 350: 105-108; Eckstein, 1985, Ann. Rev. Biochem. 54:367-402; Schaap et al., 1993, J. Biol. Chem. 268:6323-6331. Exemplary analogues of cAMP and cGMP are listed in the catalog at the website of BIOLOG Life Science Institute, Bremen, Germany, the address of which is BIOLOG.de. Preferably, the analogue of cAMP or cGMP is cell membrane permeable.

Specific analogues of cAMP useful in the present invention include, but are not limited to, db cAMP, 8-bromo-cAMP, Rp-cAMP and Sp-cAMPS. Specific analogues of cGMP useful in the present invention include, but are not limited to, db cGMP, 8-bromo-cGMP, 8-(4-chlorophenylthio) cGMP, Rp-cGMP and Sp-cGMPS.

In a specific embodiment, the concentration of db cAMP is about 2 mM, though in other specific embodiments, db cAMP concentrations of about 1 mM, or of about 2 to 4 mM can be used. It is known that db cAMP concentrations higher than about 4 mM become toxic to endothelial cells. Hence, 2 mM is considered to be the optimal concentration of db cAMP. In a preferred embodiment, the concentration of db cAMP ranges from about 1 mM to about 4 mM. In another specific embodiment, the concentration of 8-Br-cAMP is in a range of about 0.05 to 10 mM, preferably at about 1 mM. The term “about” as used herein is intended to cover the range of experimental variation.

5.3 Methods of Preservation and Use of Preserved Blood Vessels

The invention also provides a method of preserving or maintaining a blood vessel comprising contacting the blood vessel with a solution of the present invention comprising an analogue of cAMP and/or an analogue of cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α. The contacting comprises immersing, infusing, flushing, or perfusing. Other suitable procedures of contacting can be used. The method can be used wherein the blood vessel is intended for transplantation for a vascular bypass procedure, e.g., abdominal aneurysm repair, carotid endarterectomy, deep vein occlusion, popliteal aneurysm repair, or for a coronary arterial bypass (CABG). Hence, the preservation solution may be used to preserve a blood vessel or functional portion thereof prior to use in such vascular transplantation procedures.

Any known blood vessel or a functional portion thereof can be preserved ex vivo in solution of the invention, preferably prior to use as a vascular graft. The blood vessel can be an artery or a vein. Exemplary blood vessels include, but are not limited to, the renal artery, the radial artery, the internal mammary artery (also know as the internal thoracic artery), the right gastroepiploic artery, the inferior epigastric artery and the saphenous vein, or a functional portion thereof. Preferably, the blood vessel is a saphenous vein or functional portion thereof. For example, where the blood vessel graft is for a coronary arterial bypass, the blood vessel can be the internal mammary artery, the radial artery, the right gastroepiploic artery, the inferior epigastric artery and the saphenous vein, or a functional portion of the artery or vein. Preferably, the blood vessel for use as a graft is the saphenous vein or a functional portion thereof. In another example, where the graft is for abdominal aneurysm repair, carotid endarterectomy, deep vein occlusion, or popliteal aneurysm repair, the blood vessel is the renal artery or functional portion thereof, or the saphenous vein or a functional portion thereof. Preferably, the graft is isolated from the saphenous vein or a functional portion thereof.

As used herein, a “functional” portion of a blood vessel refers to a portion that is able to act as a vascular graft. The blood vessel can be isolated from and used as a vascular graft in, e.g., any mammal including primates, pigs, dogs, cats. Preferably, the blood vessel is isolated from a human, e.g., human child (less than 18 years old), or human adult (18 years or older). Preferably, the blood vessel is isolated from the patient in which it is subsequently used as a vascular graft.

One embodiment of the invention is directed to a method of preserving a blood vessel comprising contacting an isolated blood vessel or portion thereof ex vivo with a solution comprising an analogue of cAMP and/or cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α. Another embodiment is directed to a method of preserving a blood vessel comprising contacting an isolated blood vessel or portion thereof ex vivo with a solution consisting of (i) a fluid; and (ii) an analogue of cAMP and/or cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α, said fluid selected from the group consisting of heparinized blood, saline, buffered saline, or Ringer's saline each with or without heparin, the modified Columbia University solution, the Euro-Collins solution, the University of Wisconsin solution, the low-potassium dextran glucose solution and the CELSIOR™ solution. In a preferred embodiment, the method of preserving a blood vessel comprising contacting an isolated blood vessel or portion thereof ex vivo with a solution comprising an analogue of cAMP and/or cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α, and is at a temperature in the range of about 0.5 to about 10° C., preferably at 4° C., during the contacting step. In an alternative embodiment, the solution at the time of the contacting step is at a temperature in the range of about 10-25° C. In another preferred embodiment, the contacting is for a time period of not longer than 4 hours, preferably not longer than 2 or 3 hours. In another embodiment, the contacting is for a time period of about 60-90 minutes. In yet another embodiment, the temperature of the solution is at room temperature (23-25° C.) or 4° C. during the contacting step and the contacting is for a time period of about 60 to 90 minutes.

In an embodiment of the invention, after the cAMP and/or cGMP analogue has been added to the preservation solution, additional aliquots of the cAMP and/or cGMP analogue can be added to the solution in order to prevent an undesired decrease in the concentration of the analogue due to degradation during the incubation step while the blood vessel or portion thereof is being preserved. In one embodiment, the amount of the analogue first added to the solution is sufficient to form an initial concentration of about 1.5 to 3 mM of the analogue in the solution. Subsequently, preferably an equal or lesser amount of the analogue can be added (once or multiply) to the solution to replace the degraded analogue while the blood vessel or portion thereof is in contact with the preservation solution. In one embodiment, where the analogue is db cAMP, the initial concentration of the analogue is about at 2 mM. The addition of subsequent amounts of analogue to the solution preferably takes place at regular intervals during the contacting step. In one embodiment, such intervals are every 10 to 30 minutes.

Optionally, prior to use of the blood vessel or functional portion thereof as a vascular graft, the method further comprises a step of removing the solution from contact with the blood vessel or portion thereof. Preferably, the removing of the solution comprises flushing, immersing, infusing, or perfusing the blood vessel or portion thereof with a second solution that lacks the cAMP and/or cGMP analogue. Preferably, the second solution is appropriate for maintaining cardiovascular homeostasis in vivo, e.g., the solution lacks potassium. An exemplary solution is saline, Ringer's saline, or Ringer's Lactate, each with or without heparin.

In another embodiment, the present invention is directed to an isolated ex vivo blood vessel or functional portion thereof in contact with a solution comprising an analogue of cAMP and/or cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α, preferably at a temperature in the range of about 0.5 to about 10° C., more preferably 4° C. The present invention is also directed to an isolated ex vivo isolated blood vessel or functional portion thereof in contact with a solution comprising, or alternatively consisting of, an analogue of cAMP and/or an analogue of cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α. Preferably, the contacting is at a temperature in the range of about 0.5 to about 10° C., and more preferably at 4° C. Any known blood vessel or a functional portion thereof can be preserved ex vivo in solution of the invention, preferably prior to use as a vascular graft. Exemplary blood vessels include, but are not limited to, the internal mammary artery (also known as the internal thoracic artery), the radial artery, the right gastroepiploic artery, the inferior epigastric artery and the saphenous vein. As used herein, a “functional” portion of a blood vessel refers to a portion that is able to act as a vascular graft. The blood vessel can be isolated from, e.g., any mammal including primates, pigs, dogs, cats. Preferably, the blood vessel is isolated from a human, e.g., human child (less than 18 years old), or human adult (18 years or older).

In another embodiment, the invention is directed to a container containing a solution of the invention comprising the analogue of cAMP and/or cGMP and the blood vessel or functional portion thereof, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α. Preferably, the blood vessel or portion thereof is a human blood vessel or portion thereof.

In yet another embodiment, the present invention is directed to a method of using a blood vessel as a vascular graft comprising contacting an isolated blood vessel or functional portion thereof ex vivo with a solution comprising an analogue of cAMP and/or an analogue of cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α and; inserting the blood vessel into a patient so as to form a vascular graft in the patient. In one embodiment, the solution further comprises heparinized blood. Preferably, the temperature of the solution ranges from about 0.5 to about 10° C., and more preferably is about 4° C. Preferably, the contacting is for a time period not longer than four hours. The method can further comprise a step before inserting the vessel of removing the solution from contact with the blood vessel or portion thereof, wherein the removing step comprises flushing, immersing, infusing, or perfusing the blood vessel or portion thereof with a second solution that lacks the cAMP and/or cGMP analogue. Preferably, the second solution is appropriate for maintaining cardiovascular homeostasis in vivo, e.g., the solution lacks potassium. An exemplary solution is saline, Ringer's saline, or Ringer's Lactate, each with or without heparin.

Any known blood vessel or a functional portion thereof can be preserved ex vivo in a solution of the invention, preferably prior to use as a vascular graft. Exemplary blood vessels include, but are not limited to, the internal mammary artery (also known as the internal thoracic artery), the renal artery, the radial artery, the right gastroepiploic artery, the inferior epigastric artery and the saphenous vein. Preferably, the blood vessel or functional portion thereof is a saphenous vein or functional portion thereof. As used herein, a “functional” portion of a blood vessel refers to a portion that is able to act as a vascular graft. The blood vessel can be isolated from, e.g., any mammal including primates, pigs, dogs, cats. Preferably, the blood vessel is isolated from a human, e.g., human child (less than 18 years old), or human adult (18 years or older). Further, the blood vessel or portion thereof is isolated from the same patient receiving the graft, i.e., the graft is autologous.

In a particular embodiment, the analogue is db cAMP, 8-bromo cAMP, Rp-cAMP, Sp-cAMPS, db cGMP, 8-bromo cGMP, 8-(4-chlorophenylthio) cGMP (8-CPT cGMP), Rp-cGMP, Sp-cGMPS, or a mixture of the foregoing.

In specific embodiments, the solution is the modified Columbia University solution further comprising said analogue, or the Euro-Collins solution further comprising said analogue, or the University of Wisconsin solution further comprising said analogue, or the low-potassium dextran glucose solution further comprising said analogue, or the CELSIOR™ solution further comprising said analogue, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α. In a preferred embodiment, the solution is the Columbia University solution, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α. Optionally, the solution further comprises a vasodilator. Exemplary vasodilators include, but are not limited to, nitroglycerin, adenosine, pertussis toxin. Preferably, the vasodilator is cell membrane-permeable.

In yet another embodiment, the present invention is directed to a method for performing a coronary artery bypass graft in a patient comprising, removing from contact with a blood vessel or functional portion thereof a solution comprising an analogue of cAMP and/or cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α; and grafting the blood vessel or functional portion thereof into the patient so as to serve as a coronary bypass graft. Preferably, the patient is a human patient and the blood vessel or portion thereof was isolated from the same patient. Alternatively, the blood vessel is isolated from a non-human animal.

Another embodiment of the invention is directed to a pharmaceutical pack or kit comprising one or more containers filled with a solution of the invention comprising an analogue of cAMP and/or cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α. For example, the kit can comprise a container containing the low-potassium dextran glucose solution (PERFADEX™) further comprising an analogue of cAMP and/or cGMP. Optionally associated with such container(s) can be instructions for use of the kit and/or a notice in the form prescribed by a governmental agency regulating the manufacture, use or sale of pharmaceuticals or biological products, which notice reflects approval by the agency of manufacture, use or sale for human administration.

In other embodiments, the kit comprises, in a first container, a blood vessel preservation solution; and a first plurality of vials, each vial comprising a lyophilized aliquot of an analogue of cAMP and/or an analogue of cGMP. The kit can further comprise a second plurality of additional containers, equal in number to said first plurality, each additional container containing an amount of said blood vessel preservation solution sufficient to dissolve said lyophilized aliquot when added to each said vial containing said lyophilized aliquot. The kit can also further comprise an incubation dish large enough to hold a blood vessel or portion thereof immersed in said preservation solution of said first container. In a particular embodiment, the first container contains one liter of said blood vessel preservation solution. In a specific embodiment, the first plurality is at least three vials. In a specific embodiment, the first plurality of vials comprises a first vial containing an amount of the analogue sufficient to form a concentration of 1.5 to 3 mM of the analogue when combined with all of said blood vessel preservation solution in said first container. in a specific embodiment, the first plurality comprises a first vial, a second vial, and a third vial each containing an equal or lesser amount of said analogue than said first vial. In a specific embodiment, the kit further comprises a third plurality of needles and syringes, wherein, optionally, the number of syringes and needles is equal, and is the same as the number of vials in said first plurality. Optionally, the needles and syringes are preassembled. The kit can be packaged with a tray and is preferably sterile. In specific embodiments, the kits are used at room temperature (23-25° C.) or at about 4° C.

In a specific embodiment, the kit comprises (a) in a first container, a blood vessel preservation solution; (b) a first plurality of vials, each vial comprising a lyophilized aliquot of an analogue of cAMP and/or an analogue of cGMP; (c) a second plurality of additional containers, equal in number to said first plurality, each additional container containing an amount of said blood vessel preservation solution sufficient to dissolve said lyophilized aliquot when added to each said vial containing said lyophilized aliquot; (d) an incubation dish large enough to hold a blood vessel or portion thereof immersed in said preservation solution of said first container; and (e) at least one needle and at least one syringe, wherein the number of syringes and needles is equal, and is the same as the number of vials in said first plurality. In one specific embodiment, each said vial comprises a lyophilized aliquot of db cAMP. In a specific embodiment, the first plurality comprises a first vial containing an amount of said db cAMP sufficient to form a concentration of 1.5 to 3 mM of said db cAMP when combined with all of said blood vessel preservation solution in said first container. In particular embodiments, the blood vessel preservation solution can be the Euro-Collins solution, the University of Wisconsin solution, the low-potassium dextran glucose solution (PERFADEX™), the CELSIOR™ solution, the modified Columbia University solution, saline, Ringer's saline, Ringer's Lactate, heparinized saline, heparinized Ringer's saline, or heparinized Ringer's Lactate.

In an exemplary embodiment, the first container of blood vessel preservation solution has a volume of 1 liter, a first vial in the first plurality of vials contains 0.98 g of lyophilized db cAMP, and the additional containers containing an amount of the preservation solution each contain a 1 ml aliquot of the solution. The needles and syringes can be used to dissolve the lyophilized db cAMP by using an assembled needle plus syringe to take up the 1 ml of solution and deliver it to a vial in said first plurality so as to enable dissolving the aliquot of the db cAMP in the 1 ml aliquot of solution, which 1 ml reconstituted solution is then added to the 1 liter of preservation solution, resulting in an initial 2 mM concentration of db cAMP in the solution in which the blood vessel of portion thereof will be incubated. Optionally, the additional vials of lyophilized db cAMP can be likewise reconstituted with the additional containers of aliquots of solution using the additional needles and syringes and added to the solution after time intervals in order to prevent an undesired decrease in the concentration of the analogue due to degradation or hydrolysis during the incubation step while the blood vessel or portion thereof is being preserved. The above can be carried out with any analogue of cAMP or analogue of cGMP described herein.

The following series of examples are presented by way of illustration and not by way of limitation on the scope of the present invention.

### 6. EXAMPLES

6.1 In carrying out a coronary bypass using a saphenous vein as the vascular graft, the patient is first anesthetized and a portion of the saphena is excised from either leg. The excised saphenous vein is placed in contact with a preservation solution comprising an analogue of cAMP and/or cGMP in a kidney dish, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α, such that the solution is both inside and outside the vein and the dish is placed on ice. The excision in the leg is closed, and, concurrently, the chest is opened to allow access to the heart. The patient is placed on life support with a cardiac bypass machine and the heart is stopped. The saphenous vein is removed from the solution and is rinsed (flushed) with buffered saline lacking the cAMP analogue and potassium ions. The saphenous vein is cut to size for the bypass area and is grafted onto the cardiac tissue. The inserted venous segment acts as a bypass of the blocked portion of the coronary artery, and, thus, provides for a free or unobstructed flow of blood to the heart. The patient's heart is restarted and the chest is closed.

6.2 The following experimental vein graft procedure is used to assess the efficacy of a composition of the invention in ex vivo preservation of a blood vessel to be used as a vascular graft.

Vein Graft Procedure—Intrapositional Saphenous Vein Anastamosis

Female sheep, dogs or pigs are purchased from Charles River (Charles River, Mass.). The investigation will conform with the Guide for the Care and Use of Laboratory Animals published by the US National Institutes of Health (NIH Publication No. 85-23, revised 1996). The surgical procedures used are standard; animals are anesthetized by intraperitoneal injection of ketamine (50 mg/kg) and xylazine (5 mg/kg). An incision is made on the left and right legs to expose the saphenous vein and femoral artery. A segment of the saphenous vein (19 cm long) is transected after ligation at both ends with 8-0 sutures. This segment is washed with saline solution containing 100 U/ml of heparin, and stored at 25° C. for up to 2 hours in heparinized saline or in an experimental solution of heparinized saline containing a cAMP or cGMP analogue, such as db cAMP or 8-bromo cGMP.

A portion of the graft before these incubation is cut (0.5 cm) and placed in formaldehyde fixative (10%); after incubation for up to 2 hours, 0.5 cm sections are also cut and placed in formaldehyde fixative (10%), both for later immunohistochemical analysis as described, infra. After incubation in control or experimental solutions as described, a segment of the femoral artery is temporally occluded at two places with a microvascular clamp (Roboz Surgical Instrument Co., Gaithersburg, Md.), and a circular incision (of about the same size as the vein in diameter) is made. The anastamosis in loop is repaired by suturing the prepared vein into the clamped femoral artery with an 11-0 continuous suture around vein graft artery anastamosis. Contact between the instruments and the vein graft endothelium is avoided as much as possible throughout the procedure. After the vascular clamp is removed, the vein is inspected for adequacy of repair. Surgery is considered successful if strong pulsation is confirmed in both the graft and native artery without significant bleeding. If there is no pulsation or pulsations are diminished within a few minutes of restoration of blood flow, the procedure is considered a surgical failure. Cefazolin (50 mg/kg,) is administered and the skin incision is closed with a 6-0 nylon suture. Buprenorphine (2.5 mg/kg) is given subcutaneously for postoperative analgesia. The duration of the entire procedure is approximately 30 minutes. One leg in each animal is for the experimental solutions; the contralateral leg is always used for the control solution. In order to verify intimal hyperplasia, both phorbol myristate acetate (PMA, Sigma, St. Louis, Mo.) and lipopolysaccaride (LPS, Sigma, St. Louis, Mo.) at 1.0 uM is used to incubate saphenous vein segments for up to 2 hours as positive controls.

Morphology

Animals are sacrificed at various time points after surgery and perfusion-fixed using 10% formaldehyde at physiological pressure. The grafts, together with a short segment of the native femoral artery, are harvested and cut at the center. The specimens are embedded in medium (OCT compound), and frozen at −80° C. The section (5 μm) at the mid portion of each composite graft is stained with hematoxylin and eosin (H&E) or Van Gieson's elastic stain (Sigma, St. Louis, Mo.), and the degree of neointimal expansion is analyzed quantitatively using a Zeiss microscope and image analysis system (Media Cybernetics. Silver Spring, Md.). The consistency of neointimal formation in the central portion of the graft is histologically confirmed by analyzing serial sections from the center to the proximal and distal ends of the graft. The neointima of the vein graft is defined as the region between the lumen and the adventitia. Neointimal cell number is calculated by counting the number of nuclei visible in sections stained with H&E. The percentage of neointimal expansion is calculated as 100×(neointimal area/neointimal area+luminal area). These quantifications are performed by an observer blinded to the experimental circumstances. Masson Trichrome stain is performed according to the manufacture's instructions (Sigma, St. Louis, Mo.).

En Face Immunofluorescence

The procedure used in this study is similar to that reported by Zou et al., 2000, Circ Res 86:434-440 and Dietrich et al., 2000, Arterioscler Thromb Vasc Biol 20:343-352. The vein patch is retrieved 24 hours after surgery and mounted onto a glass slide with endothelium side up, and air-dried for 1 hour at room temperature. The segments are fixed in cold acetone (−20° C.) for 10 minutes and rinsed in PBS. The segments are then incubated with rat monoclonal antibody to MAC-1 (1:25, Pharmingen, San Diego, Calif.) for 30 minutes and visualized with FITC-labeled rabbit anti-rat IgG (1:25, Sigma, St. Louis, Mo.). MAC-1 positive sells are blindly counted at ×400 magnification in 10 fields of each segment.

Immunohistochemistry

Representative sections (5 mm) are immunostained with rat anti PECAM-1 (CD31) antibody (1:100, Pharmingen, San Diego, Calif.), rat anti-MAC-1 antibody (1:50, Pharmingen, San Diego, Calif.), and hamster anti-ICAM-1 antibody (1:100, Pharmingen, San Diego, Calif.). Sections are blocked with hydrogen peroxide (0.3%) in methanol for 10 min. Blocking is performed with goat serum (4%) and bovine serum albumin (1%) in PBS. Primary antibodies are added to slides, and incubated overnight at 4° C. Secondary antibodies (1:100; anti-hamster or rat IgG, Phamingen, San Diego, Calif.) are added for 30 min at room temperature. Sections are reacted with horseradish peroxidase conjugated streptavidin (1:100, Sigma, St. Louis, Mo.) for 30 min at room temperature and developed with 3.3′-diaminobenzidine (DAB substrate kit, Vector, Burlingame, Calif.).

Statistical Analysis

All data are expressed as mean ±SEM. Student's unpaired t test for a comparison between two groups, or ANOVA with post hoc analysis using the Bonferroni/Dunn test for a comparison among more than two groups are used to determine significant difference. P values of less than 0.05 are considered statistically significant. All analyses are performed using the Statview statistical package, version J5.0 (Abacus Concepts Inc., Berkeley, Calif.).

6.3 The following experiments used a murine autologous arterialized vein patch model in order to test whether the initial ischemic insult of vein grafts was linked to the later development of graft neointimal hyperplasia, and further, whether the restoration of the cAMP second messenger pathway would attenuate the development of neointimal hyperplasia.

Briefly, a segment of the external jugular vein of a mouse was grafted onto its abdominal aorta. Three weeks following the surgery, the degree of neointimal hyperplasia of the implanted graft was compared among 1) grafts without preservation, 2) grafts with 2 hour preservation (25° C.) in heparinized saline, and 3) grafts with 2 hour preservation in heparinized saline in the presence of a cAMP analog. In addition, cAMP contents of vein grafts and leukocyte adherence to the graft endothelium were assessed. The methodology is explained in greater detail below.

6.3.1 Methods

Mice and Vein Graft Procedure

Male C57BL/6J mice were purchased from Jackson Laboratories. All procedures were approved by Institutional Animal Care and Use Committee at Columbia University. The investigation conforms with the Guide for the Care and Use of Laboratory Animals published by the US National Institutes of Health (NIH Publication No. 85-23, revised 1996). The surgical procedures used were modified from those described by Shi et al., 1999, Circ Res. 84:883-890. Mice (12-16 weeks of age) were anesthetized by means of intraperitoneal injection of ketamine (50 mg/kg) and xylazine (5 mg/kg). A midline skin incision was made on the neck, and the right external jugular vein was dissected. A segment of the jugular vein (5 mm long) was transected after ligation at both ends with 8-0 sutures. This segment was opened longitudinally, washed with saline solution containing 100 U/ml of heparin, and stored at 25° C. for up to 2 hours in heparinized saline. After closing the neck incision with a 6-0 nylon suture, a midline skin incision was made on the abdomen. The abdominal aorta and the inferior vena cava were exposed. The segment of the abdominal aorta between the renal arteries and the aortic bifurcation was temporally occluded with a microvascular clamp (Roboz Surgical Instrument Co), and a longitudinal incision (of about the same length as the vein patch) was made. The defect of the aorta was repaired by suturing the prepared vein patch into the defect of the aorta with an 11-0 continuous suture around the margin of the patch. Contact between the instruments and the vein graft endothelium was avoided as much as possible throughout the procedure. After the vascular clamp was removed, the vein patch was inspected for adequacy of repair. The operation was considered successful if strong pulsation was confirmed in both the graft and native aorta without significant bleeding. If there were no pulsation or pulsations were diminished within a few minutes of restoration of blood flow, the procedure was considered a surgical failure. Cefazolin (50 mg/kg,) was administered, and the skin incision was closed with a 6-0 nylon suture. Buprenorphine (2.5 mg/kg) was given subcutaneously for postoperative analgesia. The duration of the entire procedure was approximately 50 minutes.

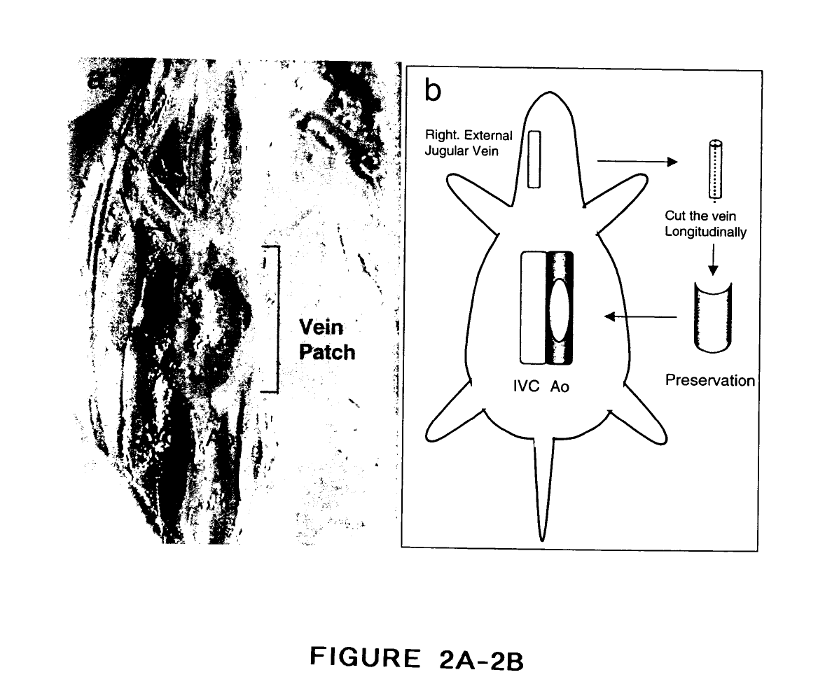

FIG. 2b demonstrates the surgical procedure. The success rate of the operation exceeded 95%. A total of 107 mice were included in this study. Ninety-three animals survived until their designated time of harvest. Two mice died intraoperatively from complications due to anesthesia, and 12 mice died postoperatively as a result of ileus (n=3), intraabdominal bleeding (n=2), graft occlusion (n=2), or unknown cause (n=5). The overall success rate was 87%.

Graft Preservation

The base preservation solution consisted of saline with heparin (100 U/ml) alone or with the following additional reagents N⁶,2′-O-dibutyryl adenosine 3′,5′-cyclic monophosphate (db-cAMP; 2 mmol/L); 8-bromoadenosine-cAMP (8-Br-cAMP; 0.1 mmol/L); and 8-Br-cAMP (0.1 mmol/L) plus Rp-cAMPS (0.25 mmol/L), the Rp isomer of adenosine 3′,5′-monophosphate (Sigma, St. Louis Mo.) (Wang et al., 2000, Circ Res. 86:982-998). The concentrations of these cAMP analogues have been shown to be the optimal dose based on previous studies (Naka et al., 1996, Circ Res. 76:773-783; Kayano et al., 1999, J Thorac Cardiovasc Surg. 118:135-144; Wang et al., 2000, Circ Res. 86:982-998) and adding cAMP analogues did not change the acidity of the base preservation solution.

Morphology

Mice were killed at various time points after surgical intervention and perfusion fixed using 10% formaldehyde at physiologic pressure. The grafts, together with a short segment of the native abdominal artery, were harvested and cut at the center. The specimens were embedded in medium (OCT compound), and frozen at −80° C. The section (5 μm) at the midportion of each composite graft was stained with hematoxylin and eosin or elastic Van Gieson stain (Sigma, St. Louis Mo.), and the degree of neointimal expansion was analyzed quantitatively with a Zeiss microscope and image analysis system (Media Cybernetics). The consistency of neointimal formation in the central portion of the graft was histologically confirmed by analyzing serial sections from the center to the proximal and distal ends of the graft (data not shown) (Shi et al., 1999, Circ Res. 84:883-890). The neointima of the vein graft was defined as the region between the lumen and the adventitia. Neointimal cell number was calculated by counting the number of nuclei visible in sections stained with hematoxylin and eosin. The percentage of neointimal expansion was calculated as follows: 100×(Neointimal area/Neointimal area+Luminal area). These quantifications were performed by an observer blinded to the experimental circumstances. Masson trichrome staining was performed according to the manufacture's instruction (Sigma, St. Louis Mo.).

cAMP Assay

Segments of inferior vena cava were taken without (n=4) or with 2 hours of preservation in the heparinized saline solution (25° C., n=4), snap-frozen in liquid nitrogen, and stored at −80° C. Tissue samples were ground to a fine powder under liquid nitrogen, weighed, and homogenized in 10 volumes of 0.1 N HCl. cAMP immunoassay was performed according to manufacturer's instruction (R&D systems Inc.).

En Face Immunofluorescence

The procedure used in this study was similar to that reported by Zou et al., 2000, Circ Res. 86:434-440 and Dietrich et al., 2000, Arterioscler Thromb Vasc Biol. 20:343-352. Vein patch was retrieved 24 hours after the operation, mounted on a glass slide with endothelium side up, and air-dried for 1 hour at room temperature. The segments were fixed in cold acetone (−20° C.) for 10 minutes and rinsed in phosphate-buffered saline. The segments were then incubated with rat monoclonal antibody to macrophage −1 antigen (MAC-1, CD116/CD18) (1:25, Pharmingen) for 30 minutes and visualized with fluorescein isothiocyanate-labeled rabbit anti-rat IgG (1:25, Sigma, St/ Louis Mo.). MAC-1-positive cells were blindly counted at 400× magnification in 10 fields of each segment.

Immunohistochemistry

Representative sections (5 μm) were immunostained with rat anti-platelet endothelial cell adhesion molecule-I (anti-PECAM-1, anti-CD31) antibody (1:100, Pharmingen), rat anti-MAC-1 antibody (1:50, Pharmingen), and hamster anti-intercellular adhesion molecule 1 (anti-ICAM-1) antibody (1:100, Pharmingen). Sections were blocked with hydrogen peroxide (0.3%) in methanol for 10 minutes. Blocking was performed with goat serum (4%) and bovine serum albumin (1%) in phosphate-buffered saline. Primary antibodies were added to slides, and incubated overnight at 4° C. Secondary antibodies (1:100; anti-hamster or anti-rat IgG, Phamingen) were added for 30 minutes at room temperature. Sections were reacted with horseradish peroxidase-conjugated streptavidin (1:100, Sigma, St. Louis Mo.) for 30 minutes at room temperature and developed with 3.3′-diaminobenzidine (DAB substrate kit, Vector).

Statistical Analysis

All data are expressed as means ±SEM. Student's unpaired t test for a comparison between two groups or analysis of variance with post hoc analysis using Bonferroni-Dunn test for a comparison among more than two groups were used to determine significant difference. All analyses were performed using the Stat-view statistical package, version J5.0 (Abacus Concepts Inc).

6.3.2 Results

Neointimal Formation in Jugular Vein Autograft

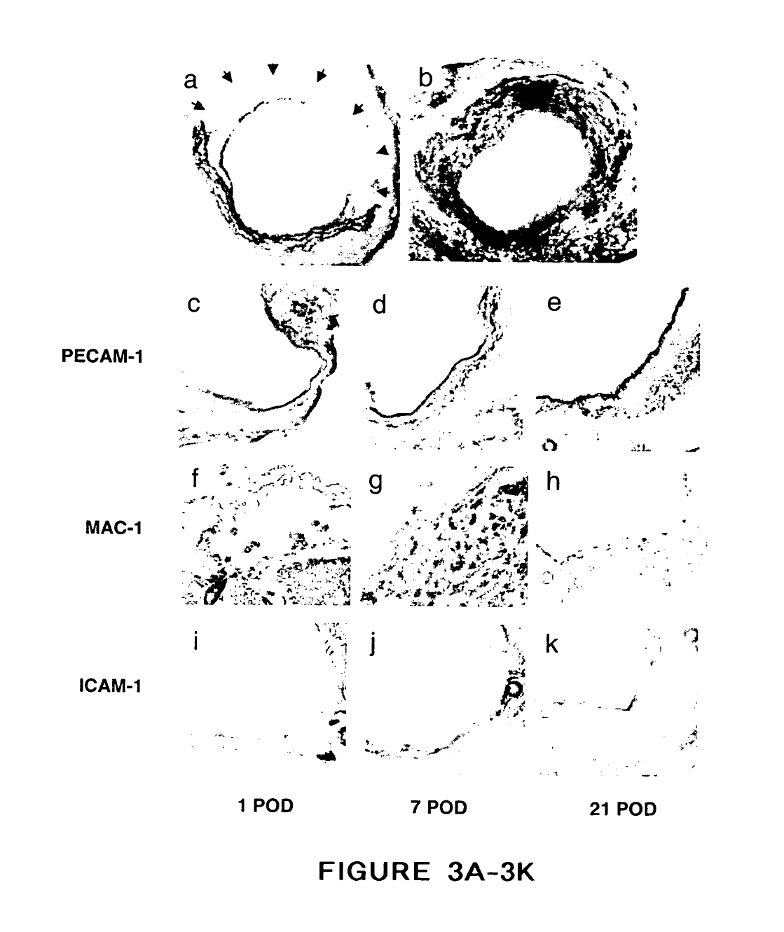

FIGS. 3a-3k show the representative histological sections of vein patch grafts that had been implanted onto the abdominal aorta immediately after harvest without preservation. Elastic Van-Gieson stain (FIG. 3a) showed that neointimal hyperplasia developed in the vein patch graft 21 days after the operation. The neointima formed primarily in association with the venous part of the composite vessel (black arrows) and not the arterial part, which is easily distinguished from the venous part by the existence of the elastic laminas (stained as black). Masson trichrome stain (FIG. 3b) indicated that the bulk of the lesion was formed by collagen (blue) and smooth muscle cells (red), which were visible by means of α-actin staining (not shown). PECAM-1 (CD31) staining indicates some varieties of continuity of the endothelium in the venous part at day 1 (FIG. 3c) which became more continuous at days 7 and 21 (FIG. 3d, 3e). MAC-1⁺ (CD11b/18) monocytes-macrophages were detected at the luminal surface at 1 day after the operation and were seen transmurally by 3 weeks (FIGS. 3f-3h). MAC-1⁺ cells were predominant in the neointima of the vein graft at postoperative day 7 and decreased their fraction in the neointima at postoperative day 21, when they were replaced by smooth muscle cells. Expression of ICAM-1, a counter-receptor for MAC-1, in the vein graft significantly increased 1 day after implantation and continued to be increased at postoperative days 7 and 21 (FIGS. 3i-3k).

Accelerated Neointimal Formation in Jugular Vein Autograft by Ischemic Preservation

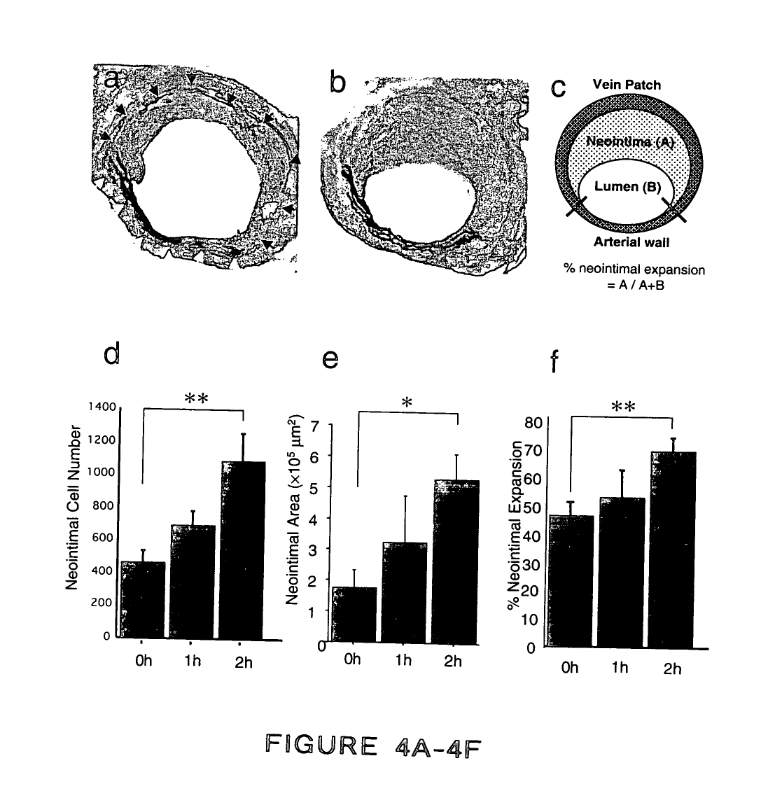

FIGS. 4a and 4b show elastic Van-Gieson-stained micrographs of a section of the jugular vein-abdominal aorta composite vessels 21 days after the operation (FIGS. 4a, 4b) and statistical data in terms of neointimal cell number (FIG. 4d), neointimal area (FIG. 4e) and percentage of neointimal expansion (FIG. 4f), which was calculated as described (FIG. 3c). Even without preservation, implanted vein grafts showed neopintimal formation 3 weeks after the operation. When vein grafts were subjected to 2 hours of preservation, they exhibited a significant increase in the severity of neointimal hyperplasia in terms of neointimal cell number (FIG. 4d; 454±77 in nonpreserved grafts vs. 1081±173 cells/neointimal area in 2-hour preserved grafts; P<0.01), neointimal area (FIG. 3e; 1.759±0.584 vs. 5.269±0.820×10⁵ μm², respectively; P<0.05) and neointimal expansion (46.1%±4.8 vs. 68.7%±9.6%, respectively; P<0.01).

cAMP Contents

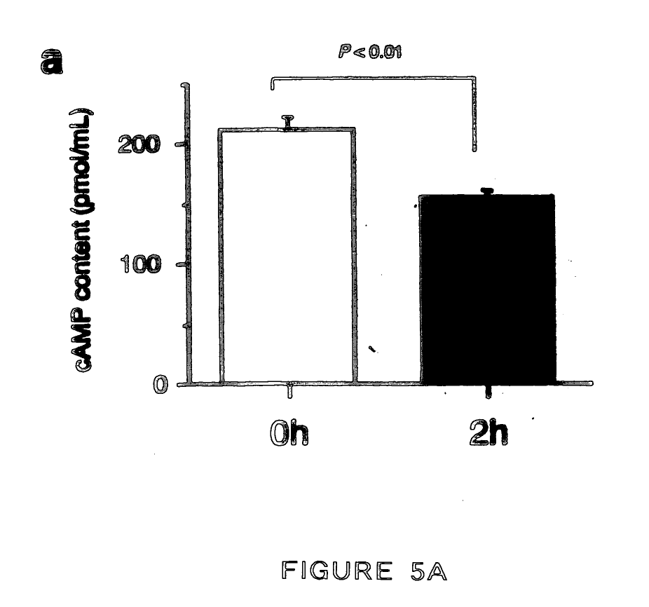

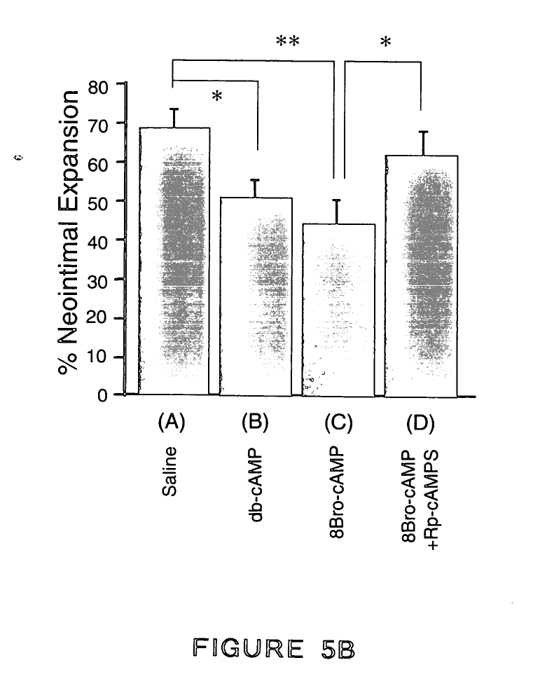

Compared with no preservation, 2 hours of preservation was associated with significantly decreased cAMP content (212±8 vs. 156±5 pmol/L, respectively; P<0.01; FIG. 5a).

Reduced Neointimal Formation by cAMP Treatment

FIG. 5b shows the effects of cAMP treatment during 2 hours of preservation on the development of neointimal expansion of vein grafts 21 days after the operation. Adding the membrane-permeable cAMP analog db-cAMP (2 mmol/L) to the preservation solution resulted in markedly diminished neointimal formation at 3 weeks (neoimtimal expansion is 68.7%±9.6% without additives [A] vs. 51.0%±4.7% with db-cAMP [B], P<0.05). To demonstrate that the beneficial effects of db-cAMP were not specific for this cAMP analog and accrue through activation by cAMP of the cAMP-dependent protein kinase, an additional series of experiments was performed. Incorporation of the membrane-permeable cAMP analog 8-Br-cAMP (0.1 mmol/L) into the preservation solution caused similar reduction of neointimal formation at 3 weeks (44.3%±5.0% [C] vs. without additives [A], P<0.01). When the same dose of 8-Br-cAMP was added to the preservation solution in the presence of additional cAMP-dependent protein kinase inhibitor, Rp-cAMPS (0.25 mmol/L), the beneficial effects of the cAMP analog with respect to diminishing neointimal formation were abolished (62.1%±6.2% [D] vs. with 8-Br-cAMP only [C], P<0.01).

Leukocyte Adhesion-Infiltration to Vein Grafts

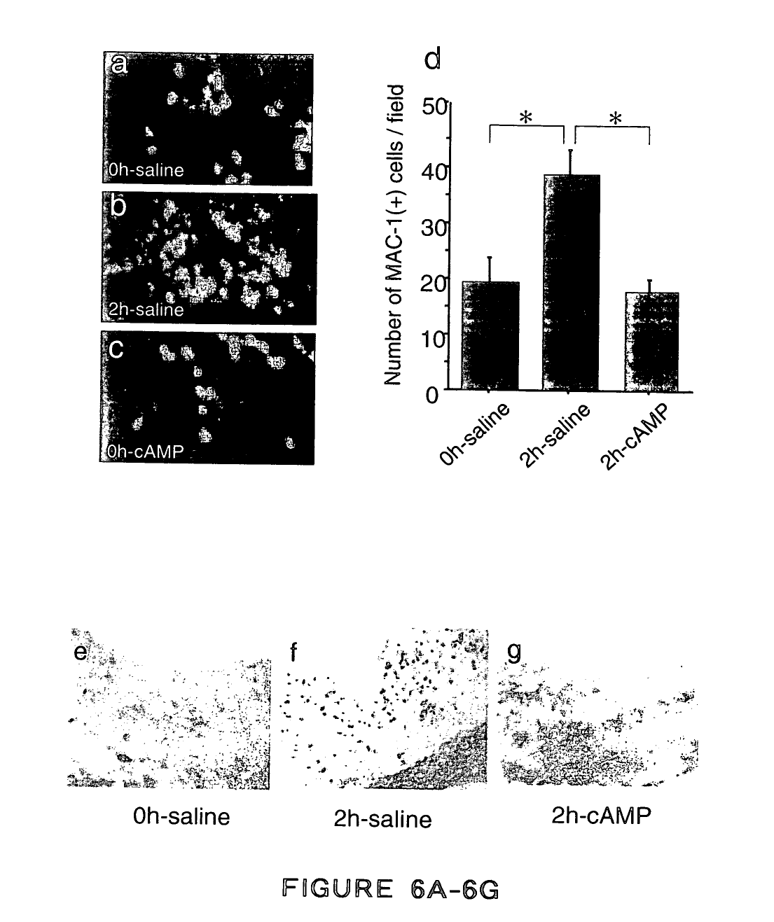

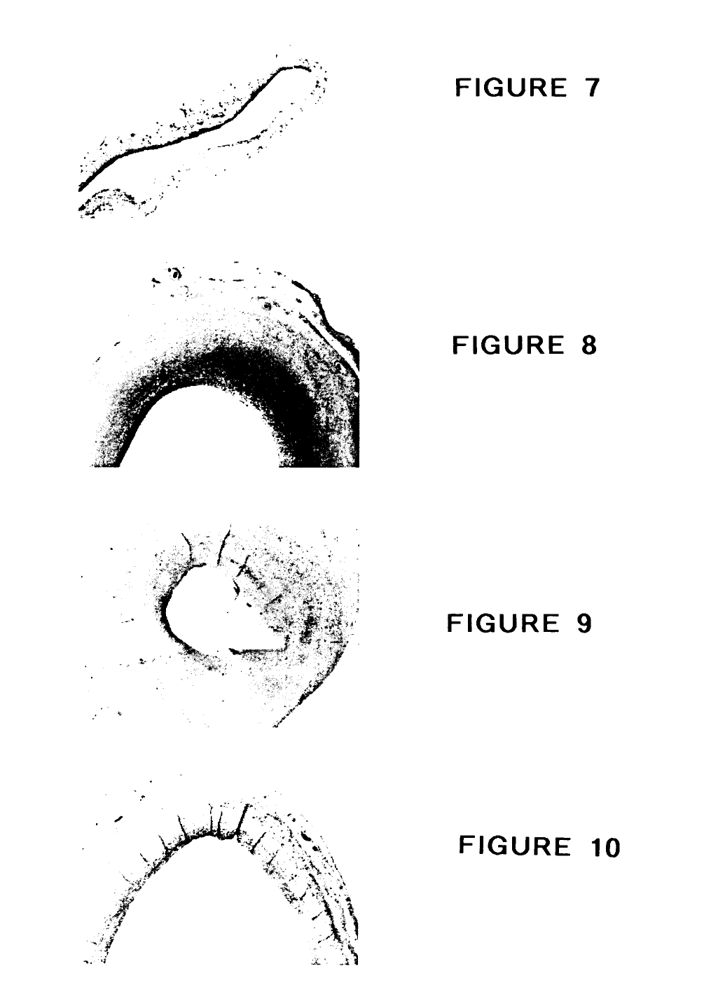

Cells adhering to the endothelial surface were visualized with a rat monoclonal antibody recognizing MAC-1⁺ leukocytes (CD11b/CD18; FIGS. 6a-6c). Significant numbers of MAC-1⁺ leukocytes (i.e., monocytes-macrophages) were observed adherent to the surface of nonpreserved vein graft segments a day after the operation (FIG. 6a). The number of MAC-1⁺ leukocytes was significantly greater in grafts exposed to 2 hours of preservation than in nonpreserved vein grafts (P<0.05; FIGS. 6b and 6d). Furthermore, adding db-cAMP to the preservation solution resulted in markedly decreased MAC-1⁺ cells in the vein grafts preserved for 2 hours (P<0.05; FIGS. 6c and 6d). Immunostaining for MAC-1 in the grafts harvested on day 21 showed that more MAC-1⁺ cells were detected in the neoimtima of 2-hour preserved grafts (FIG. 6f) compared with those in nonpreserved grafts (FIG. 6e) and 2-hour preserved grafts treated with cAMP (FIG. 6g). These results indicate that leukocyte adhesion to the endothelium is one of the earliest cellular events in vein graft disease, and the beneficial effect of cAMP on preventing vein graft disease was partly due to inhibition of leukocyte adhesion.

6.3.3 Discussion

Early endothelial cell injury has been focused on as a key factor contributing to the later development of vein graft failure (Thatte et al, 2001, Ann Thorac Surg. 72:S2245-S2252; Davies et al., 1996, J Surg Res. 66:109-114; Fulton et al., 1998, Eur J Vasc Endovasc Surg. 15:279-289; Cavallari et al., 1997, Surgery 121:64-71). Damage to endothelial cells is caused by several factors, including mechanical manipulation during harvest (Souza et al., 2002, Ann Thorac Surg. 73:1189-1195, Davies et al., 1996, J Surg Res. 66:109-114), ischemia during graft preservation (Michiels et al., 1994, Exp Cells Res. 213:43-54, Tatte et al., 2003, Ann Thorac Surg. 75:1145-1152, Michiels et al., 200, Biochim Biophys Acta 1497:1-10), and increased shear stress due to higher arterial blood pressure once implanted (Chello et al., 2003, Ann Thorac Surg. 76:453-458, Golledge et al., 1997, J Clin Invest. 99:2719-2726, Gosling et al., 1999, Circulation 99:1047-1053, Jeremy et al., 1998, Atherosclerosis 141:297-305). These factors might stimulate endothelial cells to release inflammatory mediators and growth factors with suppression of the anticoagulant cofactors, resulting in the later development of intimal hyperplasia (Davies et al., 1995, Eur J Vasc Endovasc Surg. 9:7-18, Mehta et al., 1997, Int J Cardiol. 62 Suppl 1:S55-S63, Thatte et al., 2001, Ann Thorac Surg. 72:S2245-S2252). This study focused on ischemia-induced endothelial dysfunction. Previous histochemical analyses have suggested that structural derangements in the harvested veins can be detected within two hours of storage (Thatte et al., 2001, Ann Thorac Surg. 72:S2245-S2252, Tatte et al., 2003, Ann Thorac Surg. 75:1145-1152). The level of active extracellular signal-regulated kinase in human saphenous vein is markedly increased after 50 minutes of incubation in normal saline solution (Bizekis et al. 2003, J Thorac Cardiovasc Surg. 126:659-665). Recently Thatte et al., 2003, Ann Thorac Surg. 75:1145-1152 assessed the structural and functional integrity of human saphenous vein segments stored in multiple preservation solutions using multiphoton microscopy. They showed that within 60 minutes of harvest and storage in heparinized lidocaine saline or autologous heparinized blood, calcium mobilization and nitric oxide generation were markedly diminished, with more than 90% of endothelial cells no longer viable in the vein. In this study, vein grafts preserved in heparinized saline for 2 hours showed significantly decreased tissue cAMP contents and significantly increased leukocyte adherence, followed by a marked increase in neointimal expansion 21 days after the operation compared with that seen in nonpreserved grafts. These results indicate that early endothelial damage caused by ischemic preservation might be an important contributor to the later vein graft remodeling.

cAMP is known to be an important intracellular second messenger associated with endothelial cell regulation of vascular homeostatic properties. Previous studies have shown that stimulation of the cAMP pathway prevents neointimal formation after balloon injury of the rat carotid artery and proliferation of VSMC in vitro (Indolfi et al., 2000, J Am Coll Cardiol. 36:288-293, Indolfi et al., 2001, Circ Res. 88:319-324, Bornfeldt et al., 1999, Cell Signal. 11:465-477). cAMP also inhibits tumor necrosis factor-α-induced release of IL-6 and migration of vascular smooth muscle cells cultured from human saphenous vein (Newman et al., 2003, J Surg Res. 109:57-61). In porcine saphenous vein and carotid artery interposition grafts, cAMP synthesizing capacity is significantly down-regulated at 1 month after implantation, which might be relevant to the pathophysiology of early vein graft failure (Jeremy et al., 1997, Eur J Vasc Endovasc Surg. 13:72-78, Jeremy et al., 1997, Ann Thorac Surg. 63:470-476). A recent report of the 5-year interim results of a randomized clinical trial of graft patency after coronary artery bypass surgery showed high patency of saphenous vein grafts (94%), speculating that bathing vein grafts in phosphodiesterase III inhibitor before implantation is one of the reasons associated with the very low rate of graft failure (Buxton et al., 2003, J Thorac Cardiovasc Surg. 125:1363-1371). Furthermore, it has been previously reported that in murine cardiac allografts the restoration of the cAMP pathway at the time of preservation not only improved acute allograft function but also reduced the severity of transplant-associated coronary artery disease in grafts examined 2 months after surgical intervention (Wang et al., 2000, Circ Res. 86:982-998). The donor hearts preserved for 120 minutes in the presence of db-cAMP showed significantly more nitric oxide production and less superoxide release, followed by less neointimal hyperplasia 60 days after transplantation, as compared with that seen in the control hearts preserved without the cAMP analog. In this study, on the basis of the finding that tissue cAMP contents were significantly decreased after 2 hours of preservation, it was hypothesized that the restoration of the cAMP pathway would enhance vein graft preservation, and, thus, attenuation of the later development of neointimal hyperplasia.

cAMP regulates induction of a distinct set of genes, including TNF-α, IL-6, TF, the gene encoding E-selectin, VCAM-1, and ICAM-1 (Newman et al., 2003, J Surg Res. 109:57-61, Ollivier et al., 1996, J Biol Chem. 271:2028-2035, Lalli et al., 1994, J Biol Chem. 269:1759-1762, Balyasnikova et al., 2000, J Cereb Blood Flow Metab. 20:688-699). Adhesion molecules play an important role in leukocyte adhesion to the endothelium, which is a primary step in the early stage of atherosclerosis (Stark et al., 1997, Arterioscler Thromb Vasc Biol. 17:1614-1621). Several lines of evidence have suggested that ICAM-1/MAC-1-dependent cellular interaction is involved in a number of inflammatory processes and in atherosclerosis via mononuclear cell adhesion and migration (Zou et al., 2000, Circ Res. 86:434-440, Dietrich et al., 2000, Arterioscler Thromb Vasc Biol. 20:343-352, Crook et al., 2000, Atherosclerosis 150:33-41, Vural et al., 1999, Eur J Cardiothorac Surg. 16:150-155). Zou et al., 2000, Circ Res. 86:434-440 have shown that neointimal hyperplasia of vein bypass grafts was significantly reduced in ICAM-1-deficient mice, compared with wild type animals. This is consistent with our preliminary study (data not shown). In this study, MAC-1⁺ cells adhering to the vein graft surface were significantly increased by 2 hours of preservation, and cAMP treatment markedly suppressed leukocytes adherence. In addition, ICAM-1 expression of the graft surface was increased by ischemic preservation (data not shown). Therefore, inhibition of leukocyte adhesion by reduction of ICAM-1 expression may be one of the important underlying mechanisms by which cAMP treatment suppressed neointimal expansion of vein grafts.

Several animal models have been developed to clarify the underlying mechanism of vein graft remodeling. Murine genetic models have considerable advantages over other animal systems in that they overcome the need to administer factors or their inhibitors. Zou et al. established the first murine model of vein graft arteriosclerosis wherein the external jugular vein or inferior vena cava were autografted-isografted into carotid arteries using the cuff technique (Zou et al., 2000, Circ Res 86:434-440, Dietrich et al., 2000, Am J Pathol. 157:659-669, Zou et al., 1998, Am J Pathol. 153:1301-1310). Shi et al. also reported another murine model in which a patch cut from the external jugular vein was grafted to repair a surgically created defect in the carotid artery (Shi et al., 1999, Circ Res. 84:833-890). In the current study a murine vein patch implantation model was employed with some modification of that reported by the latter. The vein patch taken from the external jugular vein was implanted onto the abdominal aorta of the same animal. This method is technically easier than that of Shi et al. and provides a means to avoid technical problems of interposition caused by size mismatch of the vein and artery. It is believed that this model represents a significant advance toward understanding the pathogenesis and treatment of vein graft disease.

In summary, our data indicate that initial ischemic insult plays an important role in the later development of vein graft neointimal hyperplasia and that stimulation of the cAMP second messenger pathway at the time of graft harvest is a potential strategy for the prevention of vein graft disease.

6.4 The following experiments were performed to determine the ability of various compounds to preserve vein grafts before implantation in a porcine vein graft model. Briefly, the vein grafts were excised from the pig, incubated in solutions containing the various test compounds, implanted back into the pig and excised after a set period of days. The excised veins were then examined for a variety of factors to determine the protective effect of the various compounds.

6.4.1 Materials and Methods

For the anti-macrophage antibody at the concentration of 2 mg/mL, the positive control tissue element were macrophages present in paraffin-embedded sections of formalin fixed porcine spleen, while the negative control tissue element for the anti-macrophage antibody was smooth muscle in paraffin-embedded sections of formalin-fixed porcine spleen. For the anti-CD54 antibody at the concentration of 50 mg/mL, the positive control material was ICAM-1/CD54 UV activated resin spot slides (prepared as a 20 mg/mL solution in water, spotted onto UV-resin slides, cross-linked to the slide by exposure to UV light, and air-dried prior to fixation). The negative control for the anti-CD54 antibody was human PTHrP 1-34 (human hypercalcemia of malignancy peptide) UV activated resin spot slides (prepared as a 20 mg/mL solution in water, spotted onto UV-resin slides, cross-linked to the slide by exposure to UV light, and air-dried prior to fixation). For anti-CD31 antibody at the dilution of 1:50, the positive control tissue element was endothelium in paraffin-embedded sections of formalin-fixed porcine spleen, while the negative control tissue element was smooth muscle in paraffinembedded sections of formalin-fixed porcine spleen. For all markers, an isotype control staining was performed using an antibody of the same immunoglobulin subclass but different antigenic specificity than the isotype control. Thus, for anti-CD54 and antimacrophages antibodies, the isotype control was mouse IgG1 (MsIgG1), and for anti-CD31 antibody the isotype control was goat IgG (GtIgG). In addition, an assay control wherein the primary antibody is omitted from the staining reaction was performed for all markers.

Antibodies and Reagents

1. Anti-macrophage antibody, Serotec, Raleigh, N.C., PAI No. A6589, Lot No. 0902.

2. Anti-CD31 antibody, Santa Cruz Biotechnology, Santa Cruz, Calif., PAI No. A5356, Lot No. A1604.

3. Anti-CD54 antibody, Serotec, Raleigh, N.C., PAI No. A6754-5, Lot No. 0304.

4. Mouse IgG1, Cappel, Durham, N.C., PAI Nos. A6466-7, Lot No. 02759.

5. Goat anti-human IgG, Jackson ImmunoResearch, West Grove, Pa., PAI No. A6539, Lot No. 60709.

6. Donkey anti-goat IgG, Jackson ImmunoResearch, West Grove, Pa., PAI No. A5728, Lot No. 60966.

7. Goat anti-mouse IgG, Jackson ImmunoResearch, West Grove, Pa., PAI No. A6355, Lot No. 60048.

8. ICAM-1/CD54, R&D Systems, Minneapolis, Minn., PAI No. A6639, Lot No. WV104091.

9. PTHrP 1-34, Sigma, St. Louis, Mo., Lot No. 034K49561.

10. Sodium phosphate, dibasic, Sigma, St. Louis, Mo., Lot No. 104K0014.

11. Potassium phosphate, monobasic, Sigma, St. Louis, Mo., Lot No. 044K0046.

12. Sodium chloride, Sigma, St. Louis, Mo., Lot No. 064K0170.

13. Albumin, bovine, Sigma, St. Louis, Mo., Lot No. 124K0597.

14. 3,3-diaminobenzadine (DAB), Sigma, St. Louis, Mo., Lot No. 054K8209.

15. ABC Elite Kit, Vector Laboratories, Burlingame, Calif., Kit Nos. K933-4, Lot No. PK-6100.

16. Normal goat serum, Vector Laboratories, Burlingame, Calif., Lot No. Q1130.

17. Normal horse serum, Vector Laboratories, Burlingame, Calif., Lot No. Q0708.

18. 10% Neutral buffered formalin (NBF), EMD, Gibbstown, N.J., Lot No. 4325.

19. Hematoxylin, Richard-Allen Scientific, Kalamazoo, Mich., Lot No. 32949.

20. Lithium carbonate, Sigma, St. Louis, Mo., Lot No. 074K3694.

21. Declere, Cell Marque, Hot Springs, Ark., Lot No. 34F.

22. Peroxidase solution, Dako, Carpinteria, Calif., Lot No. 124111.

23. Tween 20, Sigma, St. Louis, Mo., Lot No. 094K0052.

24. Peroxidase labeled polymer (anti-mouse), Dako, Carpinteria, Calif., Lot No. 063150.

25. Substrate chromogen solution, Dako, Carpinteria, Calif., Lot No. 124296.

26. Hydrogen peroxide, Sigma, St. Louis, Mo., Lot No. 043K34461.

27. Stable DAB, Invitrogen, Carlsbad, Calif., Lot No. 1251066.

28. Powdered milk, Giant, Frederick, Md., Lot No. 916.

Immunoperoxidase Staining Methods

The overall reaction sequences for immunoperoxidase stainings were as follows:

Anti-macrophage antibody: An indirect immunoperoxidase procedure was performed. Slides were treated with Declere antigen retrieval solution for 15 minutes followed by one rinse in phosphate-buffered saline (PBS) ([0.15M NaCl, pH 7.2]+0.05% Tween 20). Endogenous peroxidase activity was quenched by incubation with the peroxidase solution supplied in the Dako EnVision+ kit followed by two rinses in PBS (0.15M NaCl, pH 7.2)+0.05% Tween 20. The slides were then incubated for 20 minutes in a nonspecific protein block solution comprising 1% BSA and 1.5% normal goat serum in PBS (0.15M NaCl, pH 7.2)+0.05% Tween 20 followed by incubation with primary antibody for 1 hour. After two PBS (0.15M NaCl, pH 7.2)+0.05% Tween 20 rinses, the slides were treated with the Dako EnVision+ peroxidase labeled polymer for 30 minutes. The slides were rinsed twice with PBS (0.15M NaCl, pH 7.2)+0.05% Tween 20 and treated with Dako substrate-chromogen solution for 8 minutes. The slides were then rinsed in tap water, counterstained with hematoxylin, and blued with lithium chloride. Finally, the slides were dehydrated in increasing concentrations of alcohol, cleared with xylene, and mounted for interpretation. PBS (0.15M NaCl, pH 7.2)+0.05% Tween 20+1% BSA served as the diluent for all antibodies. All rinses were performed with PBS (0.15M NaCl, pH 7.2) supplemented with 0.05% Tween 20.

Anti-CD54 antibody: An indirect immunoperoxidase method was used. Slides were deparaffinzed, rinsed with deionized water, and treated with 3% hydrogen peroxide to block endogenous peroxidase activity. After two PBS (0.15M NaCl, pH 7.2)+0.05% Tween 20 rinses, the slides were incubated for 20 minutes in a nonspecific protein block comprising 1% BSA and 1.5% normal goat serum in PBS (0.15M NaCl, pH 7.2)+0.05% Tween 20. The slides were then incubated with primary antibody for 1 hour followed by two PBS (0.15M NaCl, pH 7.2)+0.05% Tween 20 rinses. The biotinylated secondary antibody was then added and incubated for 30 minutes. After two rinses in PBS (0.15M NaCl, pH 7.2)+0.05% Tween 20, the slides were treated with ABC reagent for 30 minutes followed by two PBS (0.15M NaCl, pH 7.2)+0.05% Tween 20 rinses. The slides were incubated with DAB substrate for 4 minutes, rinsed in tap water, counterstained with hematoxylin, and blued with lithium chloride. Finally, all slides were dehydrated in increasing concentrations of alcohol, cleared with xylene, and mounted for interpretation. PBS (0.15M NaCl, pH 7.2)+0.05% Tween 20+1% BSA served as the diluent for all antibodies and ABC reagent. All rinses were performed with phosphate buffered saline (PBS) (0.15M NaCl, pH 7.2) supplemented with 0.05% Tween 20.

Anti-CD31 antibody: An indirect immunoperoxidase procedure was performed. Slides were treated with Declere antigen retrieval solution for 15 minutes followed by two rinses in phosphate-buffered saline (PBS [0.15M NaCl, pH 7.2]+0.05% Tween 20). The slides were then treated with 3% hydrogen peroxide to block endogenous peroxidase activity. After two PBS (0.15M NaCl, pH 7.2)+0.05% Tween 20 rinses, the slides were incubated for 20 minutes in a nonspecific protein block comprising 1% BSA, 1.5% normal horse serum, and 5% milk in PBS (0.15M NaCl, pH 7.2)+0.05% Tween 20. The slides were then incubated with primary antibody for 2 hours at 37° C. followed by two PBS (0.15M NaCl, pH 7.2)+0.05% Tween 20 rinses. The biotinylated secondary antibody was then added and incubated for 30 minutes. After two rinses in PBS (0.15M NaCl, pH 7.2)+0.05% Tween 20, the slides were treated with ABC reagent for 30 minutes followed by two PBS (0.15M NaCl, pH 7.2)+0.05% Tween 20 rinses. The slides were incubated with stable DAB substrate for 6 minutes, rinsed in tap water, counterstained with hematoxylin, and blued with lithium chloride. Finally, all slides were dehydrated in increasing concentrations of alcohol, cleared with xylene, and mounted for interpretation. PBS (0.15M NaCl, pH 7.2)+0.05% Tween 20+1% BSA served as the diluent for the primary antibody and ABC reagent. The diluent for the secondary antibody also contained 1% milk. All rinses were performed with PBS (0.15M NaCl, pH 7.2) supplemented with 0.05% Tween 20.

For the anti-macrophage antibody, all slides were judged for adequacy of tissue elements and staining and read blind by the Study Pathologist to quantify the number of macrophages on the intima of the porcine venous graft tissues. The quantification was performed by counting at 400× the number of positively stained macrophages in and on the intima of each section of venous graft tissue in 10 fields and then taking the average number of macrophages per field for each section. A mean score among the four venous graft sections for each test group was calculated.

For the anti-CD31 antibody, all slides were judged for adequacy of tissue elements and staining and read blind by the Study Pathologist to quantify the degree of endothelialization of each section of venous graft tissue using the following scale: 0=no endothelialization, 1=less than 25% endothelialization, 2=25-50% endothelialization, 3=51-75% endothelialization, 4=greater than 75% endothelialization, 5=complete endothelialization. A mean score among the four venous graft sections for each test group was calculated.

For the anti-CD54 antibody, all slides were judged for adequacy of tissue elements and staining and read blind by the Study Pathologist to identify the tissue or cell type stained, frequency of staining (very rare, rare, occasional, or frequent), and intensity of staining (graded±[equivocal], 1+[weak], 2+[moderate], 3+[strong], 4+[intense], or Neg [negative]).

All slides were also stained with hematoxylin and eosin (H&E) and Movat's Pentachrome stains in preparation for histomorphometry. Calculations based on morphometry measurements were made using Microsoft Excel® software. For histomorphometry, neointimal area, luminal area, and percentage of neointimal expansion (100×[neointimal area/neointimal area+luminal area) was calculated for each venous graft section and the mean for each vessel were calculated.

6.4.2 Results/Discussion

This study tested the ability of a variety of preservation solutions to retard chronic intimal hyperplasia and protect vein grafts from restenosis and obstruction in a porcine interpositional graft model. The markers of inflammation of interest in this study were CD31 (PECAM-1), CD54 (ICAM-1), and macrophages (L1 or Calprotectin) in formalin-fixed, paraffin-embedded porcine venous graft tissues. Specifically, a porcine internal jugular vein was end to side anastamosed to the ipsilateral carotid artery, following 60 minutes of incubation in heparinized saline (negative control), Phorbol Myristate Acetate (PMA) as positive control, or experimental solutions. In addition to the detection of macrophages with either L1 or Calprotectin antibodies, anti-CD31 was used to detect endothelial cells, and Movat's Trichrome stain, together with biomorphometry was used to measure the percent of intimal expansion. For detection of CD31, an anti-CD31 antibody was applied to formalin-fixed, paraffin-embedded porcine spleen at one dilution (1:50). This dilution of anti-CD-31 antibody had strong to intense staining of splenic endothelium (positive control material). In order to detect CD54, mouse antihuman CD54 antibody was applied at one concentration (50 μg/mL) to ICAM-1/CD54 ultraviolet light (UV) activated resin spot slides. There was strong to intense staining of the ICAM-1/CD54 UV activated resin spot slides with 50 μg/mL of anti-CD54 antibody. The detection of macrophages was performed by the application of one concentration (2 μg/mL) of mouse anti-human macrophages antibody to formalin-fixed, paraffin embedded porcine spleen. There was moderate staining of macrophages with 2 μg/mL of anti-macrophages antibody. The three detecting antibodies did not specifically react with the negative control materials, non-CD31 expressing tissue elements in porcine spleen (i.e., smooth myofibers) for the anti-CD31 antibody, non-L1 expressing tissue elements in porcine spleen (i.e., smooth myofibers) for the anti-macrophages antibody, and human PTHrP 1-34 (human hypercalcemia of malignancy peptide) UV-resin activated spot slides for the anti-CD54 antibody. The negative control antibodies, mouse IgG1 (MsIgG1) for the anti-CD54 and anti-macrophages antibodies and goat IgG (GtIgG) for anti-CD31 antibody, did not specifically react with either positive control or negative control materials. The excellent specific reactions of the detecting antibodies with the positive control materials and the lack of specific reactivity with the negative control materials, as well as the lack of reactivity of the negative control antibodies, indicated that the assays were sensitive, specific, and reproducible. All slides were also stained with hematoxylin and eosin (H&E) and Movat's Pentachrome stains in preparation for histomorphometry.

The results indicated better endothelialization in venous graft tissues treated with 0.5× db-cAMP (1.0 mM), db cAMP+Milrinone+Rolipram, and Milrinone. See Table 1. This was based on the slightly higher endothelialization scores for the treated venous graft tissues compared to their control contralateral venous grafts. There were decreases in macrophage scores in venous graft tissues treated with db cAMP+Milrinone+Rolipram and 0.5× dbcAMP (1.0 mM) when compared to their respective contralateral venous graft tissues. There was no apparent staining of CD54 in any venous graft tissue. There was a large and significant increase in % neointimal expansion (% NE) for venous graft tissues treated with 0.5× db-cAMP (1.0 mM) and a slight increase in the % NE for PMA (positive control) when compared to the % NE of their contralateral venous graft tissues and to the heparinized saline negative control. These results indicated that 0.5× db-cAMP (1.0 mM) plays a role in protecting the grafted vessel from intimal hyperplasia, a major cause of vessel restenosis and occlusion by preventing macrophage adhesion to the endothelium and invasion into the intima.

TABLE 1

| | | | | | | C.% Neointimal | |
|---|---|---|---|---|---|---|---|
| | | | | | | Expansion | |
| | | A.Average Number of | | | | Movat's Trichrome | |
| | | Macrophages on/in | | B.Endothelialization | | [100 * (neointima area/ | |
| | | Intima* (400X objective) | | Score | | neointima | |
| | | MAC-1 Staining | | CD31 Staining | | area + lumen area] | |
| | | N = 10 slides | | N = 10 slides | | n = 10 slides | |
| | | Right IJV | Left IJV | Right IJV | Left IJV | Right IJV | Left IJV |
| Animal ID | Treatment | Control | Experimental | Control | Experimental | Control | Experimental |
| 4P644 | db-cAMP + Milrinone + Rolipram | 1.10 | 0.045 | 3.75 | 5.00 | 80.3 | 59.9 |
| 4P657 | db-cAMP + Milrinone + Rolipram | 1.00 | M | 4.5 | M | 75.8 | M |
| 4P661 | Hep saline | 1.05 | 1.15 | 4.75 | 4.25 | 81.0 | 82.8 |
| 4P663 | PMA | 0 | 0 | 5 | 4.5 | 76.8 | 78.9 |
| 4P664 | PMA | 1.25 | 3.55 | 5 | 5 | 79.4 | 78.8 |
| 4P665 | Thalidomide | 1.075 | 1.15 | 5 | 4.75 | 86.3 | 78.2 |
| 4P666 | Thalidomide | 1.275 | 1.45 | 4.75 | 4.5 | 74.2 | 80.0 |
| 4P668 | Lipopolysaccaride | 0.875 | M | 4.25 | M | 77.3 | M |
| 4P669 | Lipopolysaccaride | 1.175 | 1.575 | 4.75 | 4.75 | 72.0 | 77.7 |
| 5P005 | Enbrel | SO | SO | SO | SO | SO | SO |
| 5P012 | Enbrel | SO | SO | SO | SO | SO | SO |
| 5P006 | 0.5x db-cAMP | 1.10 | 0.025 | 4.25 | 5 | 77.4 | 55.6 |
| 5P007 | Milrinone (PDSIII) | 1.10 | 0 | 4.5 | 5 | 74.6 | 70.4 |
| 5P009 | Milrinone (PDSIII) | 1.60 | SO | 4 | SO | 77.0 | SO |
| 5P008 | Protractin* | 1.575 | 1.8 | 4.5 | 3.75 | 78.3 | 78.3 |
| 5P011 | Rolipram | 1.025 | M | 4.75 | M | 77.4 | M |
| 4P631 | Perfadex | 1.00 | M | 5 | M | NM | M |

SO = substantially occluded,
M = missing data point

### 7. REFERENCES CITED

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled. Such modifications are intended to fall within the scope of the appended claims.

All references, patent and non-patent, cited herein are incorporated herein by reference in their entireties and for all purposes to the same extent as if each individual publication or patent or patent application was specifically and individually indicated to be incorporated by reference in its entirety for all purposes.

What is claimed is:

1. A method of using a blood vessel as a vascular graft comprising:
(a) contacting an isolated blood vessel or functional portion thereof ex vivo with a solution comprising an analogue of cAMP and/or an analogue of cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α; and;
(b) inserting the blood vessel into a patient so as to form a vascular graft in the patient.

2. The method of claim 1, wherein the temperature of the solution ranges from about 0.5 to about 10° C.

3. The method of claim 1, wherein said contacting step is for a time period not longer than four hours.

4. The method of claim 1, which further comprises before step (b) a step of removing the solution from contact with the blood vessel or portion thereof.

5. The method of claim 4, wherein said removing step comprises flushing the blood vessel or portion thereof with a second solution lacking said analogue.

6. The method of claim 5, wherein said second solution is buffered saline or Ringer's Lactate.

7. The method of claim 1, wherein the blood vessel is a saphenous vein, a mammary artery, or a radial artery; and the vascular graft is a coronary artery bypass graft.

8. The method of claim 7, wherein the blood vessel is a saphenous vein.

9. The method of claim 1, wherein said analogue is selected from the group consisting of db cAMP, 8-bromo cAMP, Sp-cAMPS, db cGMP, 8-bromo cGMP, 8-(4-chlorophenylthio) cGMP, Sp-cGMPS, and a mixture of any two or more of the foregoing.

10. The method of claim 8, wherein the analogue is db cAMP.

11. The method of claim 1, wherein said solution further comprises heparinized blood.

12. The method of claim 1, wherein said solution further comprises buffered saline.

13. The method of claim 1, wherein said solution is the modified Columbia University solution further comprising said analogue.

14. The method of claim 1, wherein said solution is the Euro-Collins solution further comprising said analogue.

15. The method of claim 1, wherein said solution is the University of Wisconsin solution further comprising said analogue.

16. The method of claim 1, wherein said solution is the low-potassium dextran glucose solution further comprising said analogue.

17. The method of claim 1, where said solution is the CELSIOR™ solution further comprising said analogue.

18. The method of claim 1, wherein said solution further comprises nitroglycerin.

19. The method of claim 1 wherein said solution further comprises:
(a) a sugar in an amount sufficient to support intracellular function and maintenance of cellular bioenergetics;
(b) magnesium ions in an amount sufficient to support intracellular function and maintenance of cellular bioenergetics;
(c) a macromolecule of molecular weight greater than 20,000 daltons in an amount sufficient to maintain endothelial integrity and cellular viability;
(d) potassium ions in a concentration greater than about 110 mM; and
(e) a buffer in an amount sufficient to maintain the average pH of the blood vessel or portion thereof during said contacting step at about the physiologic pH value.

20. The method of claim 9, wherein the concentration of the analogue ranges from about 0.1 mM to 100 mM.

21. The method of claim 1, wherein said solution further comprises Phosphate Buffered Saline (PBS), Hanks' Balanced Salt Solution (HBSS), HBSS (Modified), Ringer's Lactate, Tyrodes buffer, Krebs buffer, Euro-Collins solution, University of Wisconsin solution, low-potassium dextran glucose solution, CELSIOR™ solution, or the modified Columbia University solution.

22. The method of claim 1, wherein the patient is a human.

23. The method of claim 22, wherein the blood vessel or portion thereof is from the patient.

24. A solution comprising an analogue of cAMP and/or an analogue of cGMP and heparinized blood, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α.

25. The solution of claim 24, wherein said analogue is selected from the group consisting of db cAMP, 8-bromo cAMP, Sp-cAMPS, db cGMP, 8-bromo cGMP, 8-(4-chlorophenylthio) cGMP, Sp-cGMPS, and a mixture of any two or more of the foregoing.

26. A solution consisting of an analogue of cAMP and/or an analogue of cGMP and heparinized blood, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α.

27. The solution of claim 26, wherein said analogue is selected from the group consisting of db cAMP, 8-bromo cAMP, Sp-cAMPS, db cGMP, 8-bromo cGMP, 8-(4-chlorophenylthio) cGMP, Sp-cGMPS, and a mixture of any two or more of the foregoing.

28. An isolated ex vivo blood vessel or functional portion thereof in contact with a solution comprising an analogue of cAMP and/or an analogue of cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α.

29. The blood vessel or portion thereof of claim 28, wherein the contacting is at a temperature in the range of about 0.5 to about 10° C.

30. The blood vessel or portion thereof of claim 28, wherein the blood vessel is a saphenous vein, a mammary artery, or a radial artery.

31. The blood vessel or portion thereof of claim 28, wherein the blood vessel is a saphenous vein.

32. The blood vessel or portion thereof of claim 28, wherein said analogue is selected from the group consisting of db cAMP, 8-bromo cAMP, Sp-cAMPS, db cGMP, 8-bromo cGMP, 8-(4-chlorophenylthio) cGMP, Sp-cGMPS, and a mixture of any two or more of the foregoing.

33. The blood vessel of portion thereof of claim 31, wherein the analogue is db cAMP.

34. The blood vessel or portion thereof of claim 32, wherein the concentration of the analogue ranges from about 0.1 mM to 100 mM.

35. The blood vessel or portion thereof of claim 28, wherein said solution comprises heparinized blood.

36. The blood vessel or portion thereof of claim 28, wherein said solution is buffered saline further comprising said analogue.

37. The blood vessel or portion thereof of claim 28, wherein said solution is the modified Columbia University solution further comprising said analogue.

38. The blood vessel or portion thereof of claim 28, wherein said solution is the Euro-Collins solution further comprising said analogue.

39. The blood vessel or portion thereof of claim 28, wherein said solution is the University of Wisconsin solution further comprising said analogue.

40. The blood vessel or portion thereof of claim 28, wherein said solution is the low-potassium dextran glucose solution further comprising said analogue.

41. The blood vessel or portion thereof of claim 28, wherein said solution is the CELSIOR™ solution further comprising said analogue.

42. The blood vessel or portion thereof of claim 28, wherein said solution further comprises:
(a) a sugar in an amount sufficient to support intracellular function and maintenance of cellular bioenergetics;
(b) magnesium ions in an amount sufficient to support intracellular function and maintenance of cellular bioenergetics;
(c) a macromolecule of molecular weight greater than 20,000 daltons in an amount sufficient to maintain endothelial integrity and cellular viability;
(d) potassium ions in a concentration greater than about 110 mM; and
(e) a buffer in an amount sufficient to maintain the average pH of the blood vessel or portion thereof at about the physiologic pH value.

43. The blood vessel or portion thereof of claim 28, which is a human blood vessel or portion thereof.

44. A container containing the blood vessel or portion thereof of claim 28.

45. A method of preserving a blood vessel comprising contacting an isolated blood vessel or functional portion thereof ex vivo with a solution comprising an analogue of cAMP and/or an analogue of cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α.

46. The method of claim 45, wherein said solution comprises heparinized blood.

47. The method of claim 45, wherein said contacting is for a time period not longer than four hours.

48. The method of claim 45, wherein the temperature of the solution ranges from about 0.5 to about 10° C.

49. The method of claim 45, which further comprises a step of removing the solution from contact with the blood vessel or portion thereof.

50. The method of claim 49, wherein said removing step comprises flushing the blood vessel or portion thereof with a second solution lacking said analogue.

51. The method of claim 45, wherein the blood vessel is a saphenous vein, a mammary artery, or a radial artery; and the vascular graft is a coronary artery bypass graft.

52. The method of claim 51, wherein the blood vessel is a saphenous vein.

53. The method of claim 45, wherein said analogue is selected from the group consisting of db cAMP, 8-bromo cAMP, Sp-cAMPS, db cGMP, 8-bromo cGMP, 8-(4-chlorophenylthio) cGMP, Sp-cGMPS, and a mixture of any two or more of the foregoing.

54. The method of claim 53, wherein the concentration of the analogue ranges from about 0.1 mM to 100 mM.

55. The method of claim 52, wherein the analogue is db cAMP.

56. The method of claim 45, wherein said solution further comprises buffered saline.

57. The method of claim 45, wherein said solution is the modified Columbia University solution further comprising said analogue.

58. The method of claim 45, wherein said solution is the Euro-Collins solution further comprising said analogue.

59. The method of claim 45, wherein said solution is the University of Wisconsin solution further comprising said analogue.

60. The method of claim 45, wherein said solution is the low-potassium dextran glucose solution further comprising said analogue.

61. The method of claim 45, wherein said solution is the CELSIOR™ solution further comprising said analogue.

62. The method of claim 45, wherein said solution further comprises:
(a) a sugar in an amount sufficient to support intracellular function and maintenance of cellular bioenergetics;
(b) magnesium ions in an amount sufficient to support intracellular function and maintenance of cellular bioenergetics;
(c) a macromolecule of molecular weight greater than 20,000 daltons in an amount sufficient to maintain endothelial integrity and cellular viability;
(d) potassium ions in a concentration greater than about 110 mM; and
(e) a buffer in an amount sufficient to maintain the average pH of the blood vessel or portion thereof during said contacting at about the physiologic pH value.

63. The method of claim 45 wherein the blood vessel or portion thereof is a human blood vessel or portion thereof.

64. A method of preserving a blood vessel comprising contacting an isolated blood vessel or portion thereof ex vivo with a solution consisting of (i) a fluid; and (ii) an analogue of cAMP and/or an analogue of cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α, said fluid selected from the group consisting of heparinized blood, buffered saline, the modified Columbia University solution, the Euro-Collins solution, the University of Wisconsin solution, the low-potassium dextran glucose solution and the CELSIOR™ solution.

65. The method of claim 64, wherein the temperature of the solution ranges from about 0.5 to about 10° C.

66. The method of claim 64, wherein said contacting is for a time period not longer than four hours.

67. The method of claim 64, which further comprises a step of removing the solution from contact with the blood vessel or portion thereof.

68. The method of claim 67, wherein said removing step comprises flushing the blood vessel or portion thereof with a second solution lacking said analogue.

69. The method of claim 64, wherein the blood vessel is a saphenous vein, a mammary artery, or a radial artery; and the vascular graft is a coronary artery bypass graft.

70. The method of claim 69, wherein the blood vessel is a saphenous vein.

71. The method of claim 64, wherein said inhibitor is selected from the group consisting of db cAMP, 8-bromo cAMP, Sp-cAMPS, db cGMP, 8-bromo cGMP, 8-(4-chlorophenylthio) cGMP, Sp-cGMPS, and a mixture of any two or more of the foregoing.

72. The method of claim 71, wherein the concentration of the analogue ranges from about 0.1 mM to 100 mM.

73. The method of claim 70, wherein the analogue is db cAMP.

74. The method of claim 64, wherein the blood vessel or functional portion thereof is a human blood vessel or functional portion thereof.

75. The method of claim 1, wherein the patient is a human.

76. The method of claim 1, wherein the contacting comprises immersing, infusing, flushing, or perfusing.

77. The method of claim 1, wherein the blood vessel is one or a combination of: the internal mammary artery, the radial artery, right gastroepiploic artery, inferior epigastric artery, or the saphenous vein.

78. The method of claim 77, wherein the blood vessel is the saphenous vein.

79. A method for performing a coronary artery bypass graft in a patient comprising,
(a) removing from contact with a blood vessel or functional portion thereof a solution comprising an analogue of cAMP and/or an analogue of cGMP, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α; and
(b) grafting the blood vessel or functional portion thereof into the patient so as to serve as a coronary bypass graft.

80. The method of claim 79, wherein the patient is a human patient.

81. The method of claim 1, 45 or 79, wherein the solution is the Columbia University solution.

82. The method of claim 1, 45 or 79, wherein the solution is also free of an inhibitor of Type V phosphodiesterase.

83. A method of using a blood vessel as a vascular graft comprising:
(a) contacting an isolated blood vessel or functional portion thereof ex vivo with a solution comprising nitroglycerin, which solution is optionally free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α; and;
(b) inserting the blood vessel into a patient so as to form a vascular graft in the patient.

84. The method of claim 83, wherein the solution lacks an analogue of cAMP and/or cGMP.

85. A solution comprising nitroglycerin and heparinized blood, which solution is free of: (i) an inhibitor of Type I phosphodiesterase, (ii) an inhibitor of Type II phosphodiesterase, (iii) an inhibitor of Type III phosphodiesterase, (iv) an inhibitor of Type IV phosphodiesterase, and (v) an inhibitor of TNF-α.

86. The solution of claim 85, wherein the solution lacks an analogue of cAMP and/or an analogue of cGMP.

87. The method of claim 1, 45 or 64, wherein subsequent amounts of the analogue are added to the solution during said contacting step.

88. The method of claim 1, 45 or 64, wherein the temperature of the solution ranges from about 0.5 to 25° C.

89. A kit comprising:
(a) in a first container, a blood vessel preservation solution; and
(b) a first plurality of vials, each vial comprising a lyophilized aliquot of an analogue of cAMP and/or an analogue of cGMP.

90. The kit of claim 89, further comprising a second plurality of additional containers, equal in number to said first plurality, each additional container containing an amount of said blood vessel preservation solution sufficient to dissolve said lyophilized aliquot when added to each said vial containing said lyophilized aliquot.

91. The kit of claim 89, further comprising an incubation dish large enough to hold a blood vessel or portion thereof immersed in said preservation solution of said first container.

92. The kit of claim 89, wherein the first container contains one liter of said blood vessel preservation solution.

93. The kit of claim 89, further wherein said first plurality is at least three vials.

94. The kit of claim 89, wherein said first plurality comprises a first vial containing an amount of said analogue sufficient to form a concentration of 1.5 to 3 mM of said analogue when combined with all of said blood vessel preservation solution in said first container.

95. The kit of claim 94, where said first plurality comprises a second vial and a third vial each containing an equal or lesser amount of said analogue than said first vial.

96. The kit of claim 89, further comprising at least one needle and at least one syringe.

97. The kit of claim 96, wherein the number of syringes and needles is equal, and is the same as the number of vials in said first plurality.

98. The kit of claim 89, wherein the blood vessel preservation solution is Ringer's saline, optionally containing heparin.

99. The kit of any of claims 89-98, which is sterile.

100. The kit of claim 99, which is at 4° C.

101. A kit comprising:
(a) in a first container, a blood vessel preservation solution;
(b) a first plurality of vials, each vial comprising a lyophilized aliquot of an analogue of cAMP and/or an analogue of cGMP;
(c) a second plurality of additional containers, equal in number to said first plurality, each additional container containing an amount of said blood vessel preservation solution sufficient to dissolve said lyophilized aliquot when added to each said vial containing said lyophilized aliquot;
(d) an incubation dish large enough to hold a blood vessel or portion thereof immersed in said preservation solution of said first container; and
(e) a third plurality of needles and syringes, wherein the number of needles and syringes is equal, and is the same as the number of vials in said first plurality.

102. The kit of claim 101, wherein each said vial comprises a lyophilized aliquot of db cAMP.

103. The kit of claim 102, wherein said first plurality comprises a first vial containing an amount of said db cAMP sufficient to form a concentration of 1.5 to 3 mM of said db cAMP when combined with all of said blood vessel preservation solution in said first container.
